(19) United States
(12) Patent Application Publication
(10) Pub. No.: US 2025/0236642 A1
(43) Pub. Date: Jul. 24, 2025

(54) PHARMACEUTICAL COMPOSITION FOR TREATMENT OR PREVENTION OF ADULT T-CELL LEUKEMIA

(71) Applicants: NEC Corporation, Tokyo (JP); The University of Tokyo, Tokyo (JP); University of the Ryukyus, Okinawa (JP); KURUME UNIVERSITY, Fukuoka (JP)

(72) Inventors: Kaoru UCHIMARU, Tokyo (JP); Kazumi NAKANO, Tokyo (JP); Yoshiko YOSHIHARA, Tokyo (JP); Takuya FUKUSHIMA, Okinawa (JP); Yuetsu TANAKA, Okinawa (JP); Kennosuke KARUBE, Okinawa (JP); Hiroaki MASUZAKI, Okinawa (JP); Koichi OSHIMA, Fukuoka (JP); Hiroaki MIYOSHI, Fukuoka (JP)

(73) Assignees: NEC Corporation, Tokyo (JP); The University of Tokyo, Tokyo (JP); University of the Ryukyus, Okinawa (JP); KURUME UNIVERSITY, Fukuoka (JP)

(21) Appl. No.: 18/838,470

(22) PCT Filed: Feb. 24, 2023
(86) PCT No.: PCT/JP2023/006691

§ 371 Date: Aug. 14, 2024

(30) Foreign Application Priority Data
Feb. 25, 2022 (JP) 2022-027480

(51) Int. Cl. C07K 7/06; A61K 39/00

(52) U.S. Cl. CPC C07K 7/06; A61K 39/00; A61K 2039/804

(57) ABSTRACT

As a technique that can be used for cancer immunotherapy of adult T-cell leukemia (ATL), provided is a pharmaceutical composition for treatment or prevention of adult T-cell leukemia, the pharmaceutical composition containing a peptide consisting of an amino acid sequence of any one of SEQ ID NOs: 1 to 68.

### INCORPORATION BY REFERENCE

This application is based upon and claims the benefit of priority from Japanese patent application No. 2022-027480, filed on Feb. 25, 2022, the disclosure of which is incorporated herein in its entirety by reference.

### TECHNICAL FIELD

The present invention relates to a pharmaceutical composition for treatment or prevention of adult T-cell leukemia (ATL). More specifically, the present invention relates to a pharmaceutical composition for treatment or prevention of ATL, the pharmaceutical composition containing a peptide consisting of a specific amino acid sequence.

### BACKGROUND ART

Development of cancer immunotherapy using a cancer antigen peptide has been advanced. For example, Patent Literature 1 discloses cancer immunotherapy using a peptide derived from glypican 3 (GPC3) which is a cancer antigen. It has been revealed that GPC3 is expressed in almost all hepatocellular carcinomas, while GPC3 is hardly expressed in normal liver, liver cirrhosis, and the like. It is also known that GPC3 is highly expressed not only in a hepatocellular carcinoma but also in melanoma, ovarian cancer, and the like.

A cancer cell elimination reaction by a cytotoxic T lymphocyte (CTL) is induced by specific recognition of a cancer antigen (HLA epitope) consisting of 8 to 11 amino acids presented in a major histocompatibility antigen (HLA) class I molecule on a surface of a cancer cell by a T cell antigen receptor (TCR) on the CTL. HLA molecules are roughly classified into a class I molecule (HLA-A type, B type, or C type) and a class II molecule (HLA-DP type, DQ type, or DR type).

Human T-cell leukemia virus type 1 (HTLV-1) is a retrovirus that primarily infects a CD4 positive T cell and causes adult T-cell leukemia (ATL).

ATL is a malignant hematologic tumor that infiltrates various organs of the whole body, and is classified into four disease types of acute type, lymphoma type, chronic type, and smoldering type. The acute type and the lymphoma type are also referred to as “aggressive ATL”, and are hematopoietic malignancies with the worst prognosis. The chronic type and the smoldering type are also referred to as “indolent ATL”, a majority which causes blast crisis during the course and has poor prognosis.

Therapies for ATL include multi-agent combination chemotherapy. However, even in the latest report, a median survival time of “aggressive ATL” is 13 months, which is not satisfactory. A patient with “indolent ATL” may survive for a long period of time even without treatment, and is often followed up without treatment until a condition deteriorates due to blast crisis or the like.

### CITATION LIST

### Patent Literature

Patent Literature 1: WO 2016/143816 A

### SUMMARY OF INVENTION

### Technical Problem

A main object of the present disclosure is to provide a technique that can be used for ATL cancer immunotherapy.

### Solution to Problem

In order to solve the above problems, the present disclosure provides the following [1] to [15].
[1]A pharmaceutical composition for treatment or prevention of adult T-cell leukemia (ATL), the pharmaceutical composition containing a peptide consisting of an amino acid sequence of any one of SEQ ID NOs: 1 to 68.
[2] The pharmaceutical composition according to [1], in which the peptide binds to one or more types of major histocompatibility antigen (HLA) molecules.
[3] The pharmaceutical composition according to [2], in which the peptide consists of an amino acid sequence of any one of SEQ ID NOs: 1 to 47 and binds to two or more types of HLA molecules.
[4] The pharmaceutical composition according to [3], in which the peptide consists of an amino acid sequence of any one of SEQ ID NOs: 1 to 33 and binds to three or more types of HLA molecules.
[5] The pharmaceutical composition according to any one of [1] to [4], in which the peptide induces a cytotoxic T lymphocyte (CTL).
[6] The pharmaceutical composition according to [5], in which the peptide consists of an amino acid sequence of any one of SEQ ID NOs: 16, 19, 20, 21, 47, and 66.
[7] The pharmaceutical composition according to any one of [1] to [6], which is in a form of a vaccine.
[8]A peptide consisting of an amino acid sequence of any one of SEQ ID NOs: 1 to 68.
[9] The peptide according to [8], which binds to one or more types of HLA molecules.
[10] The peptide according to [9], which consists of an amino acid sequence of any one of SEQ ID NO: 1 to 47 and binds to two or more types of HLA molecules.
[11] The peptide according to [10], which consists of an amino acid sequence of any one of SEQ ID NO: 1 to 33 and binds to three or more types of HLA molecules.
[12] The peptide according to any one of [8] to [11], which induces CTL.
[13] The peptide according to [12], consisting of an amino acid sequence of any one of SEQ ID NOs: 16, 19, 20, 21, 47, and 66.
[14]A method for producing an antigen-presenting cell having cytotoxic T lymphocyte inducing activity, the method including a step of bringing the peptide according to any one of [8] to [13] into contact with the antigen-presenting cell in vitro.
[15] Use of the peptide according to any one of [8] to [13] for producing a pharmaceutical composition for treatment or prevention of ATL.

### Advantageous Effects of Invention

The present disclosure provides a technique available for cancer immunotherapy of ATL.

### BRIEF DESCRIPTION OF DRAWINGS

FIG. 1 illustrates a result of stimulating peripheral blood mononuclear cells (PBMC) of an ATL patient (sample 1) with a peptide and measuring an IFN-γ production amount.

FIG. 2 illustrates a result of stimulating PBMC of an ATL patient (sample 3) with a peptide and measuring an IFN-γ production amount.

FIG. 3 illustrates a result of stimulating PBMC of an ATL patient (sample 4) with a peptide and measuring an IFN-γ production amount.

FIG. 4 illustrates a result of stimulating PBMC of an ATL patient (sample 12) with a peptide and measuring an IFN-γ production amount.

FIG. 5 illustrates a result of stimulating PBMC of an ATL patient (sample 21) with a peptide and measuring an IFN-γ production amount.

FIG. 6 illustrates a result of stimulating PBMC of an ATL patient (sample 24) with a peptide and measuring an IFN-γ production amount.

FIG. 7 illustrates a result of stimulating PBMC of an ATL patient (sample 29) with a peptide and measuring an IFN-γ production amount.

### DESCRIPTION OF EMBODIMENTS

### 1. Immunogenic Peptide

A peptide according to the present disclosure consists of an amino acid sequence of any one of SEQ ID NOs: 1 to 68.

The peptide according to the present disclosure is derived from six proteins (Casp-8, c-Myb, Fas, Helios, Wnt5a, and gp46) in which an abnormality is found in an amino acid sequence or four proteins (c-Myb, CADM1, EVC1, EVC2, p19, and tax) in which high expression is observed in an adult T-cell leukemia (ATL) patient.

When the peptide according to the present disclosure is derived from a protein in which an abnormality is found in an amino acid sequence in an ATL patient, the amino acid sequence of the peptide according to the present disclosure may include the abnormal amino acid sequence of the protein. The abnormal amino acid sequence of the protein only needs to be a sequence different from a wild-type amino acid sequence, and may be, for example, an amino acid sequence including fusion by deletion of the whole or a part of an amino acid sequence of exon (Fusion); replacement of the whole or a part of an amino acid sequence of exon (Alternative usage of exon); insertion of an amino acid sequence derived from the whole or a part of intron (Inclusion); replacement, deletion, or insertion of one or more amino acid sequences of exon; or the like.

When the peptide according to the present disclosure is derived from a protein in which high expression is observed in an ATL patient, the amino acid sequence of the peptide according to the present disclosure may consist of a part of a wild-type amino acid sequence of the protein.

Details of each of the genes Casp-8, c-Myb, Fas, Helios, Wnt5a, CADM1, EVC1, EVC2, p19, tax, and gp46 will be described below.

TABLE 1

| Gene | | Data base | ID |
|---|---|---|---|
| Casp-8 | (caspase 8) | HUGO Gene | 1509 |
| | | Nomenclature | |
| | | Commmittee | |
| | | (HGNC) | |
| c-Myb | (MYB proto-oncogene, | NGNC | 7545 |
| | transcription factor) | | |
| Fas | (Fas cell surface death | NGNC | 11920 |
| | receptor) | | |
| Helios | (IKAROS family zinc | NGNC | 13177 |
| | finger 2) | | |
| Wint5a | (Wnt family member 5A) | NGNC | 12784 |
| CADM1 | (cell adhesion molecule | NGNC | 5951 |
| | 1) | | |
| EVC1 | (EvC cillary complex | NGNC | 3497 |
| | subunit 1) | | |
| EVC2 | EvC cillary complex | NGNC | 19747 |
| | subunit 2 | | |
| p19 | | National Center | NP_955617 |
| | | for Biotechnology | |
| | | Information | |
| | | (NCBI) | |
| tax | | NCBI | BAD95658 |
| gp46 | | NCBI | NP_057865 |

Based on an abnormal amino acid sequence or a wild-type amino acid sequence of each of the above-described genes, a sequence consisting of nine amino acid residues predicted to exhibit a high binding property to a major histocompatibility antigen (HLA) molecule by a hypothesis obtained using an active learning experimental method (JP 8-151396 A) was selected. A peptide consisting of the selected amino acid sequences was synthesized, and binding ability to an HLA molecule and a cytotoxic T lymphocyte (CTL) inducibility using interferon γ (IFN-γ) production as an index were evaluated in vitro.

An amino acid sequence of a peptide and binding ability to an HLA molecule are presented in Table 2. The binding ability to an HLA molecule is indicated by a dissociation constant Kd value (−log Kd value).

In the Table, “>−3.0” indicates that a Kd value is larger than −3.0 and there is no significant binding ability.

In addition, for example, description of “>−3.37” as seen in a measured value of “allele A*02:06” of SEQ ID NO: 26 indicates that a Kd value was smaller than −3.0 and significant binding ability was confirmed, but a Kd value could not be deterministically measured and was a value between −3.37 and −3.0.

TABLE 2

| | | Measured | Measured | Measured |
|---|---|---|---|---|
| SEQ | Amino | value of | value of | value of |
| ID | acid | allele | allele | allele |
| NO. [symbol] | sequence [symbol] | A*24:02 [symbol] | A*02:01 [symbol] | A*02:06 [symbol] |
| 1 | LLLFSAAAL | −4.955161 | −6.055175 | −4.963929 |
| 2 | YVYDPPTTI | −6.23388 | −6.157512 | −5.552212 |
| 3 | YTYDPPTTI | −5.507897 | −6.474238 | −5.174326 |
| 4 | YŁYDPPTTI | −6.159959 | −6.234504 | −5.868 |
| 5 | AQAPPTAQL | −5.752784 | −6.017318 | −5.848128 |
| 6 | ALAPPTAQL | −5.329069 | −6.576803 | −6.406524 |
| 7 | ALLAPAVIL | −5.962306 | −6.666211 | −5.845153 |
| 8 | KFLEAFHEV | −6.877614 | −5.930552 | −5.80648 |
| 9 | CLOEHOWQL | −5.865796 | −6.664691 | −6.324304 |
| 10 | ATLAPAVIL | −5.204216 | −5.555678 | −6.115734 |
| 11 | AALDDERTL | −4.87668 | −5.053362 | −6.072933 |
| 12 | ALPKIRARV | −6.180589 | −5.660504 | −4.779625 |
| 13 | LMEEHGATL | −5.223294 | −4.917532 | −5.67827 |
| 14 | SLVLTWAAL | −5.311076 | −5.627463 | −6.242613 |
| 15 | VLLQQFQTA | −5.550844 | −4.713793 | −5.468897 |
| 16 | LLITRKSFI | −6.638063 | −5.131336 | −4.625881 |
| 17 | LMITRKSFI | −6.012118 | −5.203411 | −4.532591 |
| 18 | ALPAPHLTL | −6.733862 | −5.993502 | −5.518169 |
| 19 | VMSHHGEKL | −6.100718 | −5.07986 | −4.297741 |
| 20 | IALETPVWI | −5.482722 | −5.109722 | 5.518556 |
| 21 | IQYSSFHNL | −6.531752 | −5.28612 | −5.600099 |
| 22 | ALQFLIPRL | −4.96382 | −5.900906 | −5.83809 |
| 23 | LLNPNROTI | −6.220197 | −5.240723 | −3.528096 |
| 24 | MLPQTPSHL | −7.144786 | −5.578598 | −3.962718 |
| 25 | ATAPPTAQL | −4.535162 | −4.995851 | −6.164923 |
| 26 | ALRPAPALL | −6.401676 | −6.111296 | >−3.37 |
| 27 | AVLAPAVEL | −4.805983 | −5.152921 | −5.153776 |
| 28 | CIDEHQWQL | −3.478245 | −4.852411 | −5.250628 |
| 29 | LHTRKSFI | −5.602002 | −4.107437 | −3.637789 |
| 30 | KVITRKSFI | −5.459937 | −4.010582 | −3.614017 |
| 31 | ALQPPCPNE | −5.306697 | −5.128578 | −4.636105 |
| 32 | YQLSPPITW | −5.763919 | −3.455134 | −5.749343 |
| 33 | TLTAWQHGL | −4.695985 | −5.953639 | −5.847836 |
| 34 | ISAYRTOWI | −4.999866 | >−3 | −3.137432 |
| 35 | FSNDFGQSL | >−3 | −3.900423 | −4.714286 |
| 36 | CMIVHOGTI | −5.53543 | >−3 | −4.372437 |
| 37 | SSFEFFEEA | >−3 | −6.49437 | −7.423289 |
| 38 | HMAKVIEFL | −6.03476 | −5.686323 | >−3 |
| 39 | CTDEHQWOL | >−3 | −5.042581 | −6.795019 |
| 40 | LIEEHGATL | >−3 | −4.49659 | −4.814942 |
| 41 | LLEEHGATL | >−3 | −5.601954 | −5.303772 |
| 42 | LTEEHGATL | >−3 | −4.085562 | −4.923626 |
| 43 | LVEEHGATL | >−3 | −4.179687 | −4.861812 |
| 44 | YFFTVIFSF | −6.774954 | >−3 | >−4.4 |
| 45 | LTITRKSFI | −4.82322 | >−3 | −3.789322 |
| 46 | LAITRKSFI | −5.118736 | >−3 | −3.040449 |
| 47 | EAIDGYITL | −5.53598 | >−3 | −4.722986 |
| 48 | TVLVPPRNL | −5.270565 | >−3 | >−3 |
| 49 | AYRTCWIFS | −5.418114 | >−3 | >−3 |
| 50 | AQISAYRTC | >−3 | >−3 | −4.085741 |
| 51 | GTHLDAGTV | >−3 | >−3 | −3.661209 |
| 52 | YPGWHSTTI | −5.660838 | >−3 | >−3 |
| 53 | ETLQFIDSM | >−3 | >−3 | −3.37066 |
| 54 | VLPPARHST | >−3 | −5.111948 | >−3 |
| 55 | SSLDPPKVL | −5.349805 | >−3 | >−3 |
| 56 | SLPFSPSQF | −6.998552 | >−3 | >−3 |
| 57 | FIDSDSSSW | −4.701162 | >−3 | >−3 |
| 58 | CDLSSFEFF | −4.095109 | >−3 | >−3 |
| 59 | ATGDCSSFI | >−3 | −4.080581 | >−3 |
| 60 | LPFSPSQFL | −3.348308 | >−3 | >−3 |
| 61 | LAEEHGATL | >−3 | >−3 | −4.495261 |
| 62 | SLLITRKSF | −5.84291 | >−3 | >−3 |
| 63 | LHITRKSFI | −4.757461 | >−3 | >−3 |
| 64 | HMTSSERPF | −4.164965 | >−3 | >−3 |
| 65 | GYITCERPF | −6.52078 | >−3 | >−3 |
| 66 | VFGRVMQID | −5.412266 | >−3 | >−3 |
| 67 | VMQIDGVQP | >−3 | >−3 | −3.410122 |
| 68 | GRVMQIDGV | −4.631654 | >−3 | >−3 |
| 69 | SLPNEKQTS | >−3 | >−3 | >−3 |
| 70 | ILSDHQQSQ | >−3 | >−3 | >−3 |
| 71 | STGESAQTS | >−3 | >−3 | >−3 |
| 72 | GSPDEFSND | >−3 | >−3 | >−3 |
| 73 | SPDEFSNDF | >−3 | >−3 | >−3 |
| 74 | GILSDHQQS | >−3 | >−3 | >−3 |
| 75 | THLDAGTVE | >−3 | >−3 | >−3 |
| 76 | LPPARHSTI | >−3 | >−3 | >−3 |
| 77 | ARHSTIPLV | >−3 | >−3 | >−3 |
| 78 | IFADVSSST | >−3 | >−3 | >−3 |
| 79 | LHEEHGATL | >−3 | >−3 | >−3 |
| 80 | GYITLGKPH | >−3 | >−3 | >−3 |
| 81 | ASPSHMTSM | >−3 | >−3 | >−3 |

Peptides of SEQ ID NOs: 1 to 4, 23, and 48 are derived from CADM1.

Peptides of SEQ ID NOs: 5, 6, 24, 25, 36, 37, 52 to 60, and 76 to 78 are derived from c-Myb.

Peptides of SEQ ID NOs: 7 to 10, 26 to 28, 38, and 39 are derived from EVC1.

Peptides of SEQ ID NOs: 11 to 15, 40 to 43, 61, and 79 are derived from EVC2.

Peptides of SEQ ID NOs: 16, 17, 29, 30, 44 to 46, 62, and 63 are derived from Fas.

Peptides of SEQ ID NOs: 18 and 31 are derived from gp46.

Peptides of SEQ ID NOs: 19, 478, 64, 65, 80, and 81 are derived from Helios.

A peptide of SEQ ID NO: 20 is derived from p19.

Peptides of SEQ ID NOs: 21, 22, 32, and 33 are derived from tax.

Peptides of SEQ ID NOs: 34, 35, 49 to 51, and 69 to 75 are derived from Casp-8.

Peptides of SEQ ID NOs: 66 to 68 are derived from Wnt5a.

The peptide according to the present disclosure binds to one or more types (subtypes) of HLA molecules. The peptides according to the present disclosure binds to preferably two or more, more preferably three or more types of HLA molecules.

Peptides of SEQ ID NOs: 1 to 33 exhibit a binding property to all of a product of an HLA-A*24:02 gene (HLA-A*24:02 molecule), a product of an HLA-A*02:01 gene (HLA-A*02:01 molecule), and a product of an HLA-A*02:06 gene (HLA-A*02:06 molecule).

Peptides of SEQ ID NOs: 34 to 47 exhibit a binding property to any two or more of an HLA-A*24:02 molecule, an HLA-A*02:01 molecule, and an HLA-A*02:06 molecule.

Peptides of SEQ ID NOs: 48 to 68 exhibit a binding property to any one of an HLA-A*24:02 molecule, an HLA-A*02:01 molecule, and an HLA-A*02:06 molecule.

HLA subtypes to which the peptide according to the present disclosure can bind are not limited to HLA-A*24:02, HLA-A*02:01, and HLA-A*02:06. However, since these HLA subtypes cover about 85% of Oriental people including Japanese and about 55% of Western people, it is considered that a multi-HLA peptide of the present invention has a wide patient coverage in immunotherapy and the like.

Furthermore, the peptide according to the present disclosure induces CTL. In particular, peptides of SEQ ID NOs: 16, 19, 20, 21, 47, and 66 exhibit CTL inducing activity in an ATL patient.

The peptide according to the present disclosure has an HLA binding property and immunogenicity. As used in the present specification, “immunogenicity” means being capable of inducing an immune reaction (immunity inducibility), for example, increasing cytotoxic T lymphocyte (CTL) inducing activity of an antigen-presenting cell, and further increasing cytotoxic activity of CTL against a cancer cell.

In addition, as used in the present specification, “CTL induction” means that in vitro or in vivo, the peptide according to the present disclosure is presented on a surface of an antigen-presenting cell to induce or proliferate CTL that specifically recognizes a certain antigen, to differentiate a naive T cell into an effector cell having ability (cytotoxic activity) to kill a target cell such as a cancer cell, and/or to increase cytotoxic activity of the CTL.

The CTL inducing activity can be measured by evaluating cytokine (for example, IFN-γ) production by CTL. For example, CTL inducing activity may be measured by evaluating an increase in cytokine-producing cells induced from precursor cells by antigen-presenting cells such as peripheral blood mononuclear cells stimulated with the peptide according to the present disclosure using a known high-sensitivity immunoassay such as an Enzyme-Linked ImmunoSpot (ELISPOT) method or an Enzyme-Linked ImmunoSorbent Assay (ELISA) method.

The cytotoxic activity of CTL can also be measured by a known method such as a ⁵¹Cr release method.

When the activity is significantly increased as compared with a control, for example, when the activity is increased by 5% or more, 10% or more, 20% or more, preferably 50% or more, it can be evaluated that immunity or CTL is induced.

The peptide according to the present disclosure has a high HLA binding property and high CTL inducibility, and is therefore strongly expected to be useful as a cancer vaccine. Applications of the peptide according to the present disclosure to various immunotherapies, particularly to a dendritic cell therapy are also expected. In addition, the peptide according to the present disclosure can bind to a plurality of HLA types. Therefore, the peptide according to the present disclosure can provide a cancer vaccine, a dendritic cell therapy, and the like that cover an extremely wide ATL patient group.

In the peptide according to the present disclosure, amino acid residues constituting amino acid sequences of SEQ ID NOs: 1 to 68 or a part thereof may be modified as long as the peptide retains immunogenicity. The amino acid sequences represented by SEQ ID NOs: 1 to 68 are intended to be amino acid sequences in a state of being presented on an antigen-presenting cell. However, when the peptide is directly administered into a body, the peptide may undergo a change such as digestion of a terminal of the peptide in a digestive organ or the like depending on an administration route. Therefore, the peptide according to the present disclosure may be present in a form of a precursor having one or more amino acid residues and the like added to an N-terminal and/or a C-terminal before being incorporated into an antigen-presenting cell such that the amino acid residues represented by SEQ ID NOs: 1 to 68 are retained when the peptide binds to a predetermined HLA class I molecule on the antigen-presenting cell.

In the peptide according to the present disclosure, one or several amino acid residues may be replaced, inserted, deleted, or added, and/or modified in such a manner as glycosylation, side chain oxidation, and/or phosphorylation as long as the peptide has desired immunogenicity.

In the present specification, “amino acid” is used in its broadest meaning, and includes an artificial amino acid variant and a derivative in addition to a natural amino acid. In the present specification, examples of the amino acid include: a natural proteinaceous L-amino acid; a D-amino acid; a chemically modified amino acid such as an amino acid variant or a derivative; a natural non-proteinaceous amino acid such as norleucine, β-alanine, or ornithine; and a chemically synthesized compound having properties known in the art, the properties being characteristics of the amino acid. Examples of the non-natural amino acid include an α-methylamino acid (such as α-methylalanine), a D-amino acid, a histidine-like amino acid (such as β-hydroxy-histidine, homohistidine, α-fluoromethyl-histidine, or α-methyl-histidine), an amino acid having an extra methylene in a side chain (“homo” amino acid), and an amino acid in which a carboxylic acid functional group amino acid in a side chain is replaced with a sulfonic acid group (such as cysteic acid).

Regarding replacement of an amino acid residue and the like, those skilled in the art can appropriately replace an amino acid residue constituting the peptide according to the present disclosure in consideration of regularity of a peptide sequence that exhibits a binding property to HLA (J. Immunol., 152: p 3913, 1994; Immunogenetics, 41: p 178, 1995; J. Immunol., 155: p 4307, 1994).

More specifically, in a case of a peptide that binds to an HLA-A*24:02 molecule, a second position amino acid constituting the peptide may be replaced with tyrosine, phenylalanine, methionine, or tryptophan, and/or an amino acid at a C-terminal may be replaced with phenylalanine, leucine, isoleucine, tryptophan, or methionine.

In addition, in a case of a peptide that binds to an HLA-A*02:01 molecule, a second position amino acid may be replaced with leucine or methionine, and/or an amino acid at a C-terminal may be replaced with valine or leucine.

Furthermore, in a case of a peptide that binds to an HLA-A*02:06 molecule, a second position amino acid may be replaced with valine or glutamine, and/or an amino acid at a C-terminal may be replaced with valine or leucine.

Peptides of SEQ ID NOs: 3 and 4 derived from CADM1 are each obtained by introducing a second position amino acid replacement into a peptide of SEQ ID NO: 2.

Peptides of SEQ ID NOs: 6 and 25 derived from c-Myb are each obtained by introducing a second position amino acid replacement into a peptide of SEQ ID NO: 5.

Peptides of SEQ ID NOs: 10, 27, 28, and 39 derived from EVC1 are each obtained by introducing a second position amino acid replacement into each of peptides of SEQ ID NOs: 7 and 9.

Peptides of SEQ ID NOs: 40 to 43, 61, and 79 derived from EVC2 are each obtained by introducing a second position amino acid replacement into a peptide of SEQ ID NO: 13.

Peptides of SEQ ID NOs: 17, 29, 30, 45, 46, and 63 derived from Fas are each obtained by introducing a second position amino acid replacement into a peptide of SEQ ID NO: 16.

The peptide according to the present disclosure can be produced using a technique known to those skilled in the art. For example, the peptide according to the present disclosure may be artificially synthesized by a solid phase method such as an Fmoc method or a tBoc method or a liquid phase method. In addition, a desired peptide may be produced by expressing a polynucleotide encoding the peptide according to the present disclosure or a recombinant vector containing the polynucleotide. In addition, any one of the peptides thus obtained can be identified using a technique known to those skilled in the art. Any one of the peptides thus obtained can be identified using, for example, an Edman degradation method or a mass spectrometry method.

### 2. Pharmaceutical Composition

A pharmaceutical composition for treatment or prevention of ATL according to the present disclosure contains, as an active component, for example, a peptide consisting of amino acid residues of SEQ ID NOs: 1 to 68.

The peptide according to the present disclosure induces CTL by being presented on an antigen-presenting cell, and this induced CTL injures a cancer cell. Therefore, the active component of the pharmaceutical composition according to the present disclosure is not limited to the peptide according to the present disclosure, and may be a component capable of directly or indirectly inducing CTL, for example, a polynucleotide encoding the peptide or a vector containing the polynucleotide, an antigen-presenting cell that presents a complex of the peptide and an HLA molecule on a surface or an exosome secreted from the antigen-presenting cell, or a combination thereof. Examples of the antigen-presenting cell to be used include a macrophage and a dendritic cell, and it is preferable to use a dendritic cell having high CTL inducibility.

Other components known to be used for cancer treatment, for example, a chemokine, a cytokine, a tumor necrosis factor, a chemotherapeutic agent, and the like may be contained in the pharmaceutical composition according to the present disclosure. A dose of the peptide may be, for example, about 1 to 10 mg per day when a patient is an adult. However, the dose varies depending on the age of a patient, the weight of the patient, an administration method, and the like, and is therefore appropriately determined by those skilled in the art.

Although not intended to be limited, the pharmaceutical composition according to the present disclosure is considered to be useful for killing a cancer cell and the like by the following mechanism of action. The pharmaceutical composition according to the present disclosure is administered to an ATL patient, whereby the peptide in the pharmaceutical composition is presented on a surface of an antigen-presenting cell in a state of being binding to an HLA molecule. When CTL recognizes such a peptide on the antigen-presenting cell, CTL is activated, proliferates, and circulates in the whole body. When CTL specific to the peptide enters a cancer tissue, the CTL recognizes the same peptide derived from a specific cancer antigen naturally binding to an HLA molecule on a surface of a cancer cell, and kills the cancer cell. This function can contribute to treatment of cancer.

The pharmaceutical composition according to the present disclosure can be used not only for treatment of cancer but also for prevention of cancer. For example, by administering the pharmaceutical composition according to the present disclosure to a healthy human body, CTL is induced and the induced CTL remains in the body. Therefore, when a cancer cell develops, the CTL can injure the cancer cell. Similarly, by administering the pharmaceutical composition according to the present disclosure to a human body after cancer treatment, recurrence of cancer may be prevented.

The pharmaceutical composition according to the present disclosure can be dissolved in a water-soluble solvent, formulated in a form of a pharmaceutically acceptable salt, and administered to a patient. Examples of such a form of a pharmaceutically acceptable salt include a form buffered at a physiological pH in a form of a physiologically acceptable water-soluble salt, for example, in a form of a salt of sodium, potassium, magnesium, or calcium. In addition to the water-soluble solvent, a water-insoluble solvent can also be used, and examples of such a water-insoluble solvent include an alcohol such as ethanol or propylene glycol.

In addition, a formulation containing the pharmaceutical composition of the present embodiment may contain agents for various purposes, and examples of such an agent include a preservative and a buffer. Examples of the preservative include sodium bisulfite, sodium bisulfate, sodium benzalkonium thiosulfate chloride, chlorobutanol, thimerosal, phenylmercuric acetate, phenylmercuric nitrate, methylparaben, polyvinyl alcohol, phenylethyl alcohol, ammonia, dithiothreitol, and beta-mercaptoethanol. Examples of the buffer include sodium carbonate, sodium borate, sodium phosphate, sodium acetate, and sodium bicarbonate. These agents can be present in an amount by which pH of the system can be maintained between 2 to 9, preferably 4 to 8.

A dosage form of the pharmaceutical composition according to the present disclosure is not particularly limited, but when the pharmaceutical composition is used as a form of a vaccine, examples of the dosage form include an injection (muscle, subcutaneous, or intradermal), an oral formulation, and a nasal formulation. When the pharmaceutical composition according to the present disclosure is in a form of a vaccine, the vaccine may be a mixed cocktail vaccine containing a plurality of types of active components. For example, such a vaccine may contain any two or more of the peptides of SEQ ID NO: 1 to 68 or a plurality of types of active components in combination with other active components.

In addition, the vaccine may be an inactive component-containing vaccine containing a component that is a component other than the pharmaceutical composition, is not active by itself, and has an effect of further enhancing the effect of the pharmaceutical composition as the vaccine. Examples of the inactive component include an adjuvant and a toxoid. Examples of the adjuvant include a precipitating type adjuvant such as aluminum hydroxide, aluminum phosphate, or calcium phosphate, and an oily type adjuvant such as Freund's complete adjuvant or Freund's incomplete adjuvant, but are not intended to be limited thereto.

When the pharmaceutical composition according to the present disclosure is present in a form of a vaccine, the pharmaceutical composition is preferably administered into a body by injection or infusion, for example, by intradermal, subcutaneous, intravenous, or intramuscular administration, or by transdermal inhalation or inhalation from a mucous membrane of a nose, a pharynx, or the like. A single dose of the pharmaceutical composition can be set between an amount by which cytotoxic T lymphocytes can be significantly induced and an amount by which a significant number of non-cancer cells are not injured.

The pharmaceutical composition according to the present disclosure is intended not only for administration to a human body but also for use outside the body. More specifically, the pharmaceutical composition according to the present disclosure may be used in order to stimulate an antigen-presenting cell in vitro or ex vivo to increase CTL inducing activity. For example, a case where the pharmaceutical composition according to the present disclosure is used for cancer dendritic cell therapy will be exemplified. The pharmaceutical composition according to the present disclosure can be administered to a patient by previously being brought into contact with an antigen-presenting cell derived from a patient in need of treatment or prevention of cancer, such as a dendritic cell, and then returning the antigen-presenting cell to the patient's body. The peptide contained in the pharmaceutical composition can be introduced into the antigen-presenting cell by, for example, a lipofection method or an injection method. When a polynucleotide encoding the peptide according to the present disclosure is used in such an application, the polynucleotide can be introduced into the antigen-presenting cell by a technique known in the art. For example, the antigen-presenting cell derived from the patient may be transformed in vitro with a polynucleotide of interest or a vector encoding the polynucleotide by a lipofection method, an electroporation method, a microinjection method, a cell fusion method, a DEAE dextran method, or a calcium phosphate method.

### 3. Method for Producing Antigen-Presenting Cell

A method for producing an antigen-presenting cell according to the present disclosure includes a step of bringing peptides consisting of amino acid sequences of SEQ ID NOs: 1 to 68 into contact with the antigen-presenting cell in vitro.

It is considered that a peptide used in the production method according to the present disclosure binds to an HLA class I molecule on a surface of an antigen-presenting cell and is presented to CTL as an antigen peptide, thereby inducing CTL activity of the antigen-presenting cell. Therefore, what is brought into contact with the antigen-presenting cell is not limited to the peptide according to the present disclosure, and may be a component capable of directly or indirectly inducing CTL, for example, a polynucleotide encoding the peptide or a vector containing the polynucleotide, an antigen-presenting cell that presents a complex of the peptide and an HLA molecule on a surface or an exosome secreted from the antigen-presenting cell, or a combination thereof. Examples of the antigen-presenting cell to be used include a macrophage and a dendritic cell, and it is preferable to use a dendritic cell having high CTL inducibility.

An antigen-presenting cell produced by the production method according to the present disclosure is intended to be used not only as an active component of the pharmaceutical composition or an immunity inducer but also for immunotherapy or the like. For example, a case where the antigen-presenting cell is used for cancer dendritic cell therapy will be exemplified. The antigen-presenting cell produced can be administered to a patient by previously being brought into contact with an antigen presenting cell derived from a patient in need of immune induction, such as a dendritic cell having low CTL inducibility, and then returning the antigen presenting cell to the patient's body. The peptide according to the present disclosure can be introduced into the antigen-presenting cell by, for example, transfection via liposome (lipofection method) or an injection method. When a polynucleotide encoding the peptide according to the present disclosure is used in such an application, the polynucleotide can be introduced into the antigen-presenting cell by a technique known in the art. For example, the antigen-presenting cell derived from the patient may be transformed in vitro with a polynucleotide of interest or a vector encoding the polynucleotide by a lipofection method, an electroporation method, a microinjection method, a cell fusion method, a DEAE dextran method, or a calcium phosphate method.

### Example

### 1. Extraction of Amino Acid Sequence

Procedures of prediction, experiment, and evaluation in the present Example were performed based on an active learning experimental plan described in WO 2006/004182 A, and the following steps were repeated as a whole to construct a rule.

(1) One trial of a lower-level learning algorithm described later is performed. That is, a plurality of hypotheses are expressed from random resampling of accumulated data, and a point having the largest variance of a prediction value for a randomly expressed question candidate point (peptide) is selected as a question point to be tested.

(2) The peptide of the selected question point is produced by a synthesis and purification method described later, and actual binding ability is measured by an experiment described later and added to the accumulated data.

By performing such an active learning method, it was possible to reduce the number of binding experiments that are originally required to be performed on a peptide consisting of nine amino acid residues for all 500 billion (=20⁹) or more candidate substances of an HLA binding peptide.

According to the rule as described above, the amino acid sequences represented by SEQ ID NOs: 1 to 81 were extracted. Specifically, an amino acid sequence having a predicted binding score of 3 or more in terms of a −log Kd value with a product of an HLA-A*24:02 gene (HLA-A*24:02 molecule), a product of an HLA-A*02:01 gene (HLA-A*02:01 molecule), and a product of an HLA-A*02:06 gene (HLA-A*02:06 molecule) was selected.

TABLE 3

| | | Predicted | Predicted | Predicted |
|---|---|---|---|---|
| SEQ | Amino | value of | value of | value of |
| ID | acid | allele | allele | allele |
| NO [symbol] | sequence [symbol] | A*24:02 [symbol] | A*02:01 [symbol] | A*02:06 [symbol] |
| 1 | LLLFSAAAL | 5.3954 | 5.6122 | 5.4844 |
| 2 | YVYDPPTTI | 5.2483 | 5.3878 | 5.3878 |
| 3 | YTYDPPTTI | 5.2849: | 5.341 | 5.0547 |
| 4 | YLYDPPTTI | 5.7999 | 6.2767 | 5.4006 |
| 5 | AQAPPTAQL | 4.947 | 4.5297 | 4.9742 |
| 6 | ALAPPTAQL | 5.5137 | 6.3111 | 5.7006 |
| 7 | ALLAPAVLL | 5.4892 | 5.6076 | 5.3213 |
| 8 | KFLEAFHEV | 5.4475 | 5.2151 | 5.8664 |
| 9 | CLDEHQWQL | 5.0394 | 5.1216 | 4.8845 |
| 10 | ATLAPAVEL | 4.9621 | 5.56 | 5.7903 |
| 11 | AALDDERTI | 5.5918 | 5.0362 | 5.0734 |
| 12 | ALPKIRARV | 5.3585 | 5.5682 | 4.6902 |
| 13 | LMEEHGATL | 5.3357 | 5.7236 | 4.8107 |
| 14 | SLVLTWAAL | 5.3297 | 5.357 | 5.0377 |
| 15 | VLLQQFQTA | 4.9267 | 5.265 | 5.2232 |
| 16 | LLITRKSFI | 4.9325 | 4.9786 | 4.865 |
| 17 | LMITRKSPI | 5.1761 | 4.903 | 4.771 |
| 18 | ALPAPHLTL | 6.1275 | 4.9379 | 4.701 |
| 19 | VMSHHGEKL | 5.1725 | 4.3364 | 4.1436 |
| 20 | IALETPVWI | 5.0997 | 5.3423 | 5.108 |
| 21 | IQYSSFHNL | 5.9032 | 4.6083 | 5.3579 |
| 22 | ALQFLIPRL | 5.1527 | 5.0659 | 5.1431 |
| 23 | LLNPNRQTI | 5.7314 | 5.0241 | 4.4348 |
| 24 | MLPQTPSHL | 5.5978 | 5.3224 | 5.0358 |
| 25 | ATAPPTAQL | 4.9892 | 5.2349 | 5.8204 |
| 26 | ALRPAPALL | 5.8917 | 5.1698 | 4.7738 |
| 27 | AVLAPAVIL | 5.1909 | 5.5813 | 5.5063 |
| 28 | CIDEHQWQL | 5.0194 | 5.3401 | 5.8291 |
| 29 | LIITRKSFI | 4.9699 | 4.1916 | 4.2501 |
| 30 | LVITRKSFI | 4.6453 | 4.4255 | 4.7705 |
| 31 | ALQPPCPNL | 5.2403 | 4.8818 | 5.1202 |
| 32 | YQLSPPITW | 5.2881 | 4.4009 | 5.1576 |
| 33 | TLTAWQHGL | 4.9203 | 4.8909 | 4.4191 |
| 34 | ISAYRTCWI | 3.8267 | 4.0678 | 3.9299 |
| 35 | FSNDFGQSL | 4.5854 | 4.3705 | 3.7956 |
| 36 | CMIVHQGTI | 5.1993 | 5.301 | 4.593 |
| 37 | SSFEFFEEA | 3.4109 | 4.9633 | 4.5643 |
| 38 | HMAKVIEFL | 6.0024 | 4.9312 | 4.5233 |
| 39 | CTDEHQWQL | 4.84 | 5.1202 | 6.1246 |
| 40 | LIEEHGAIL | 4.6546 | 5.122 | 4.5639 |
| 41 | LLEEHGATL | 5.1337 | 5.6417 | 5.0018 |
| 42 | LTEEHGATL | 4.5876 | 4.7387 | 4.7278 |
| 43 | LVEEHGATL | 4.9208 | 5.1172 | 4.8716 |
| 44 | YFFTVIFSF | 6.2086 | 4.0936 | 4.1279 |
| 45 | LTITRKSFI | 4.847 | 3.6475 | 4.2049 |
| 46 | LAITRKSFI | 4.2534 | 4.4135 | 4.6998 |
| 47 | EAIDGYITL | 4.7563 | 3.8951 | 4.5883 |
| 48 | TVLVPPRNL | 5.0304 | 4.6162 | 4:2269 |
| 49 | AYRTCWIFS | 5.4627 | 3.5213 | 3.2048 |
| 50 | AQISAYRTC | 4.3869 | 3.7377 | 4.5628 |
| 51 | GTHLDAGTV | 3.5569 | 4.0198 | 4.9566 |
| 52 | YPGWHSTTI | 4.8158 | 4.7384 | 4.406 |
| 53 | ETLQFIDSM | 4.3549 | 3.4982 | 4.424 |
| 54 | VLPPARHST | 4.4968 | 4.2431 | 5.3975 |
| 55 | SSLDPPKVL | 4.5358 | 4.8509 | 4.4647 |
| 56 | SLPFSPSQF | 5.7771 | 5.2367 | 4.401. |
| 57 | FIDSDSSSW | 4.5959 | 3.721 | 4.2743 |
| 58 | CDLSSFEFF | 5.5317 | 3.6074 | 3.879 |
| 59 | ATGDCSSFI | 5.0873 | 4.3748 | 4.6378 |
| 60 | LPFSPSQFL | 4.3883 | 4.2067 | 4.3114 |
| 61 | LAEEHGATL | 4.4813 | 5.0977 | 4.6394 |
| 62 | SLLITRKSF | 5.2341 | 4.2079 | 4.7207 |
| 63 | LHITRKSFI | 4.5085 | 4.0391 | 4.3093 |
| 64 | HMTSSERPF | 5.5799 | 3.8544 | 3.558 |
| 65 | GYITCERPF | 6.4771 | 3.2797 | 3.1197 |
| 66 | VFGRVMQID | 4.4473 | 3.1074 | 3.5402 |
| 67 | VMQIDGVQP | 3.6744 | 4.6173 | 4.3009 |
| 68 | GRVMQIDGV | 3.0177 | 4.5084 | 4.1937 |
| 69 | SLPNEKQTS | 4.1512 | 4.4894 | 4.4993 |
| 70 | ILSDHQQSQ | 4.5813 | 4.1746 | 4.0446 |
| 71 | STGESAQTS | 3.6495 | 4.277 | 4.5435 |
| 72 | GSPDEFSND | 4.3578 | 3.7201 | 3.8972 |
| 73 | SPDEFSNDF | 4.3723 | 3.4784 | 3.6062 |
| 74 | GILSDHQQS | 4.0334 | 4.0314 | 4.5669 |
| 75 | THLDAGTVE | 3.8107 | 4.1005 | 3.7226 |
| 76 | LPPARHSTI | 4.9947 | 4.4871 | 4.3819 |
| 77 | ARMSTIPLV | 3.6597 | 4.5648 | 5.2325 |
| 78 | IFADVSSST | 4.9891 | 3.7176 | 3.9608 |
| 79 | LHEEHGATL | 4.7524 | 4.6916 | 4.3323 |
| 80 | GYITLGKPH | 4.6376 | 3.3894 | 3.2538 |
| 81 | ASPSHMTSM | 4.7099 | 3.7652 | 4.2798 |

### 2. Peptide Synthesis and Purification

Peptides having amino acid sequences of SEQ ID NOs: 1 to 81 were manually synthesized by a solid phase method of Merrifield using a Fmoc amino acid. After deprotection, reverse phase HPLC purification was performed using a C18 column to set a purity to 95% or more. Identification of a peptide and confirmation of a purity of the peptide were performed by MALDI-TOF mass spectrometry (AB SCIEX MALDI-TOF/TOF5800). Quantification of a peptide was performed by Micro BCA assay (Thermo Scientific) using BSA as a standard protein.

### 3. Experiment of Binding of Peptide to HLA-A Molecule

### (1) HLA-A*24:02 Molecule

Measurement of binding ability of a peptide to an HLA-A*24:02 molecule, which is a product of an HLA-A*24:02 gene, was performed using C1R-A24 cells expressing an HLA-A*24:02 molecule (Cells created by Professor Masafumi Takiguchi, Kumamoto University, was provided by Assistant Professor Masaki Yasukawa, Ehime University, with permission).

First, the C1R-A24 cells were exposed to acidic conditions at pH 3.3 for 30 seconds to dissociate and remove an endogenous peptide originally binding to an HLA-A*24:02 molecule and light chain β2m commonly associated with an HLA class I molecule. After neutralization, purified β2m was added to the C1R-A24 cells, and the mixture was added to a peptide dilution series and incubated on ice for four hours. Staining was performed using a fluorescently-labeled monoclonal antibody 17A12 that recognizes an association (MHC-pep) of three members of an HLA-A*24:02 molecule, a peptide, and β2m which had been reassociated during this time.

Thereafter, the number of MHC-peps for each C1R-A24 cell (proportional to a fluorescence intensity of the fluorescent antibody) was quantitatively measured using a fluorescence cell analyzer FACScan (Becton-Dickinson). From an average fluorescence intensity per cell, a binding/dissociation constant Kd value between an HLA-A*24:02 molecule and a peptide was calculated by a method disclosed in an article (Udaka et al., Immunogenetics, 51, 816-828, 2000) by the present inventor.

### (2) HLA-A*02:01 Molecule

Measurement of binding ability of a peptide to an HLA-A*02:01 molecule, which is a product of an HLA-A*02:01 gene, was performed using a cell line T2 (purchased from ATCC) expressing an HLA-A*02:01 molecule.

T2 cells and purified β2m were added to a serial dilution series of a peptide whose binding ability was to be measured, and then the mixture was incubated at 37° C. for four hours. HLA-A*02:01 molecules whose expression level increased in a peptide concentration-dependent manner by this time point were stained using an association-type specific fluorescently labeled monoclonal antibody BB7.2.

Thereafter, the amount of fluorescence per cell was measured with a flow cytometer, and a dissociation constant Kd value was calculated by the method disclosed in the article (Udaka et al., Immunogenetics, 51, 816-828, 2000) by the present inventor.

### (3) HLA-A*02:06 Molecule

Measurement of binding ability of a peptide to an HLA-A*02:06 molecule, which is a product of an HLA-A*02:06 gene, was performed using RA2.6 cells (a cell line newly created at Kochi University) obtained by introducing cDNA of the HLA-A*02:06 gene into RMAS, which is a mouse transporter associated with antigen processing (TAP)-deficient cell line.

First, the RA2.6 cells were cultured overnight at 26° C., a peptide dilution series was added to accumulation of the HLA-A*02:06 molecules to which no peptide was binding on a cell surface, and bonding was performed at 26° C. for 60 minutes.

Thereafter, by performing culture at 35° C. for four hours, an empty HLA-A*02:06 molecule to which no peptide was binding was denatured, and a steric structure was lost. A fluorescently labeled monoclonal antibody BB7.2 that specifically recognizes a peptide binding type HLA-A*02:06 molecule was added thereto, and the mixture was incubated on ice for 20 minutes to stain the cells.

Thereafter, the amount of fluorescence per cell was measured with a flow cytometer, and a dissociation constant Kd value was calculated by the method described in the literature (Udaka et al., Immunogenetics, 51, 816-828, 2000).

### (4) Results

The dissociation constant Kd values are presented in Table 4. When the Kd value (−log Kd value) is smaller than −3, it can be determined that there is a binding property.

TABLE 4

| | | Measured | Measured | Measured |
|---|---|---|---|---|
| SEQ | Amino | value of | Ivalue of | value of |
| ID | acid | allele | allele | allele |
| NO. [symbol] | sequence [symbol] | A*24:02 [symbol] | A*02:01 [symbol] | A*02:06 [symbol] |
| 1 | LLLFSAAAL | −4.955161 | −6.055175 | −4.963929 |
| 2 | YVYQPPTTI | −6.23388 | −6.157512 | −5.552212 |
| 3 | YTYDPPTTI | −5.507897 | −5.474238 | −6.174326 |
| 4 | YLYDPPTTI | −6.159959 | −6.234504 | −5.868 |
| 5 | AQAPPTAQL | −5.752784 | −6.017318 | −5.848128 |
| 6 | ALAPPTAQL | −5.329069 | −6.576803 | −6.406524 |
| 7 | ALLAPAVEL | −5.962306 | −6.666211 | −5.845153 |
| 8 | KFLEAFHEV | −6.877614 | −5.930552 | −5.80648 |
| 9 | CLDEHQWQL | −5.865796 | −6.664691 | −6.324304 |
| 10 | ATLAPAVEL | −5.204216 | −5.555678 | −6.115734 |
| 11 | AALDQLRTL | −4.87668 | −5.053362 | −6.072933 |
| 12 | ALPKIRARV | −6.180589 | −5.660504 | −4.779625 |
| 13 | LMEEHGATL | −5.223294 | −4.917532 | −5.67827 |
| 14 | SLVLTWAAL | −5.311076 | −5.627463 | −6.242613 |
| 15 | VLLQQFQTA | −5.550844 | −4.713793 | −5.468897 |
| 16 | LLITRKSFI | −6.638063 | −5.131336 | −4.625881 |
| 17 | LMITRKSFI | −6.012118 | −5.203411 | −4.532591 |
| 18 | ALPAPHLTL | −6.733862 | −5.993502 | −5.518169 |
| 19 | VMSHHGEKL | −6.100718 | −5.07986 | −4.297741 |
| 20 | IALETPVWI | −5.482722 | −5.109722 | −5.518556 |
| 21 | IQYSSFHNL | −6.531752 | −5.28612 | −5.600099 |
| 22 | ALQFLIPRL | −4.96382 | −5.900906 | −5.83809 |
| 23 | LLNPNRQTI | −6.220197 | −5.240723 | −3.528096 |
| 24 | MLPQTPSHL | −7.144786 | −5.578598 | −3.962718 |
| 25 | ATAPPTAQL | −4.535162 | −4.995851 | −6.164923 |
| 26 | ALRPAPALL | −6.401676 | −6.111296 | >−3.37 |
| 27 | AVLAPAVIL | 4.805983 | −5.152921 | −5.153776 |
| 28 | CIDEHQWQL | −3.478245 | −4.852411 | −5.250628 |
| 29 | LIITRKSFI | −5.602002 | −4.107437 | −3.637789 |
| 30 | LVITRKSFI | −5.459937 | −4.010582 | −3.614017 |
| 31 | ALQPPCPNL | −5.306697 | −5.128578 | −4.636105 |
| 32 | YQLSPPITW | −6.763919 | −3.455124 | −5.749343 |
| 33 | TLTAWQHGL | −4.695985 | −5.953639 | −5.847836 |
| 34 | ISAYRTCWI | −4.999866 | >−3 | −3.137432 |
| 35 | FSNDFGQSL | >−3 | −3.900423 | −4.714286 |
| 36 | CMIVHQGTI | −5.53543 | >−3 | −4.372437 |
| 37 | SSFEFFEEA | >−3 | −6.49437 | −7.423289 |
| 38 | HMAKVIEFL | −6.03476 | −5.686323 | >−3 |
| 39 | CTDEHQWQL | >−3 | −5.042581 | −6.795019 |
| 40 | LIEEHGATL | >−3 | −4.49659 | −4.814942 |
| 41 | LLEEHGATL | >−3 | −5.601954 | −5.303772 |
| 42 | LTEEHGATL | >−3 | −4.085562 | −4.923626 |
| 43 | LVECHGATL | >−3 | −4.179687 | −4.861812 |
| 44 | YFFTVIFSF | −6.774954 | >−3 | >−4.4 |
| 45 | LTITRKSFI | −4.82322 | >−3 | −3.789322 |
| 46 | LAITRKSFI | −5.118736 | >−3 | −3.040449 |
| 47 | EAIDGYITL | −5.53598 | >−3 | −4.722986 |
| 48 | TVEVPPRNL | −8.270565 | >−3 | >−3 |
| 49 | AYRTCWIFS | −5.418114 | >−3 | >−3 |
| 50 | AQISAYRTC | >−3 | >−3 | −4.085741 |
| 51 | GTHLDAGTV | >−3 | >−3 | −3.661209 |
| 52 | YPGWHSTTI | −5.660838 | >−3 | >−3 |
| 53 | ETLQFIDSM | >−3 | >−3 | −3.37066 |
| 54 | VLPPARHST | >−3 | −5.111948 | >−3 |
| 55 | SSLDPPKVL | −5.349805 | >−3 | >−3 |
| 56 | SLPFSPSQF | −6.998552 | >−3 | >−3 |
| 57 | FIDSDSSSW | −4.701162 | >−3 | >−3 |
| 58 | CDLSSFEFF | −4.095109 | >−3 | >−3 |
| 59 | ATGQCSSFI | >−3 | −4.080581 | >−3 |
| 60 | LPFSPSQFL | −3.348308 | >−3 | >−3 |
| 61 | LAEEHGATL | >−3 | >−3 | −4.495261 |
| 62 | SLLITRKSF | −5.84291 | >−3 | >−3 |
| 63 | LHITRKSFI | −4.757461 | >−3 | >−3 |
| 64 | HMTSSERPF | −4.164965 | >−3 | >−3 |
| 65 | GYITCERPF | −6.52078 | >−3 | >−3 |
| 66 | VFGRVMQID | −5.412266 | >−3 | >−3 |
| 67 | VMQIDGVQP | >−3 | >−3 | −3.410122 |
| 68 | GRVMQIDGV | −4.631654 | >−3 | >−3 |
| 69 | SLPNEKQTS | >−3 | >−3 | >−3 |
| 70 | ILSDHQQSQ | >−3 | >>3 | >−3 |
| 71 | STGESAQTS | >−3 | >−3 | >−3 |
| 72 | GSPDEFSND | >−3 | >−3 | >−3 |
| 73 | SPDEFSNDF | >−3 | >−3 | >−3 |
| 74 | GILSDHQQS | >−3 | >−3 | >−3 |
| 75 | THLDAGTVE | >−3 | >−3 | >−3 |
| 76 | LPPARHSTI | >−3 | >−3 | >−3 |
| 77 | ARHSTIPLV | >−3 | >−3 | >−3 |
| 78 | IFADVSSST | >−3 | >−3 | >−3 |
| 79 | LHEEHGATL | >−3 | >−3 | >−3 |
| 80 | GYITLGKPH | >−3 | >−3 | >−3 |
| 81 | ASPSHMTSM | >−3 | >−3 | >−3 |

Peptides of SEQ ID NOs: 1 to 33 exhibited a binding property to all of the HLA-A*24:02 molecule, the HLA-A*02:01 molecule, and the HLA-A*02:06 molecule.

Peptides of SEQ ID NOs: 34 to 47 exhibited a binding property to any two or more of the HLA-A*24:02 molecule, the HLA-A*02:01 molecule, and the HLA-A*02:06 molecule.

Peptides of SEQ ID NOs: 48 to 68 exhibited a binding property to any one of the HLA-A*24:02 molecule, the HLA-A*02:01 molecule, and the HLA-A*02:06 molecule.

Peptides of SEQ ID NOs: 69 to 81 exhibited no binding property to any one of the HLA-A*24:02 molecule, the HLA-A*02:01 molecule, and the HLA-A*02:06 molecule, contrary to expectation.

### 4. Peptide Immune Induction Test

Peripheral blood mononuclear cells (PBMC) separated from an ATL patient were cultured and stimulated with a peptide, and an IFN-γ production amount was measured by ELISA.

A suspension of PBMC (2×10⁶ cell/ml) was seeded in a 96 well plate (100 to 200 μl/well), and cultured in a medium containing 5 μg/ml of a peptide (ASF-104, autologous serum 2%, IL-2 20 U/ml) for two to three days.

The cells were washed. Thereafter, the cells were resuspended in the same medium, 5 μg/ml of the peptide was added thereto, and the cells were further cultured for three to five days. PBMC that had not been subjected to peptide stimulation was used as a control.

A culture supernatant was collected and subjected to measurement of an IFN-γ production amount by ELISA (Human IFN-γ ELISA MAX Deluxe Set, manufactured by BioLegend, Inc.).

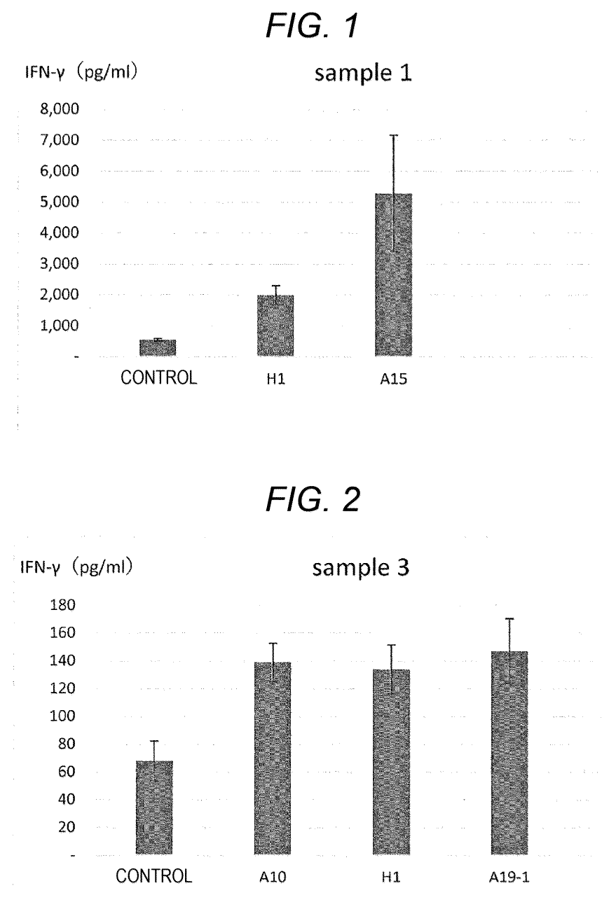

As results of a representative ELISA assay, results for seven patients are illustrated in FIGS. 1 to 7.

FIG. 1 is a result for a patient with HLA-A*24:02 in one of alleles (sample 1).

FIG. 2 is a result for a patient with HLA-A*02:06 in one of alleles (sample 3).

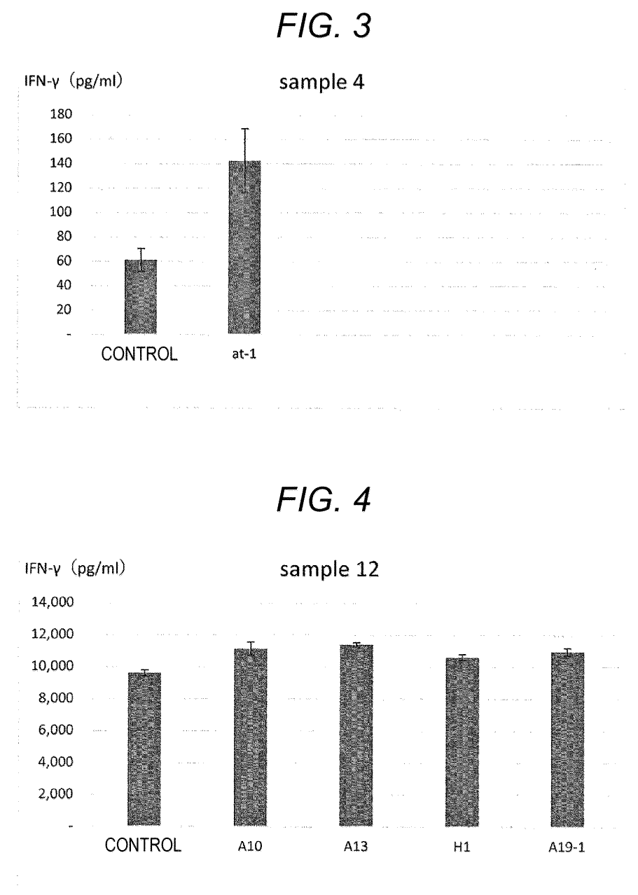

FIG. 3 is a result for a patient with HLA-A*02:06 in one of alleles (sample 4).

FIG. 4 is a result for a patient with HLA-A*02:01 in one of alleles (sample 12).

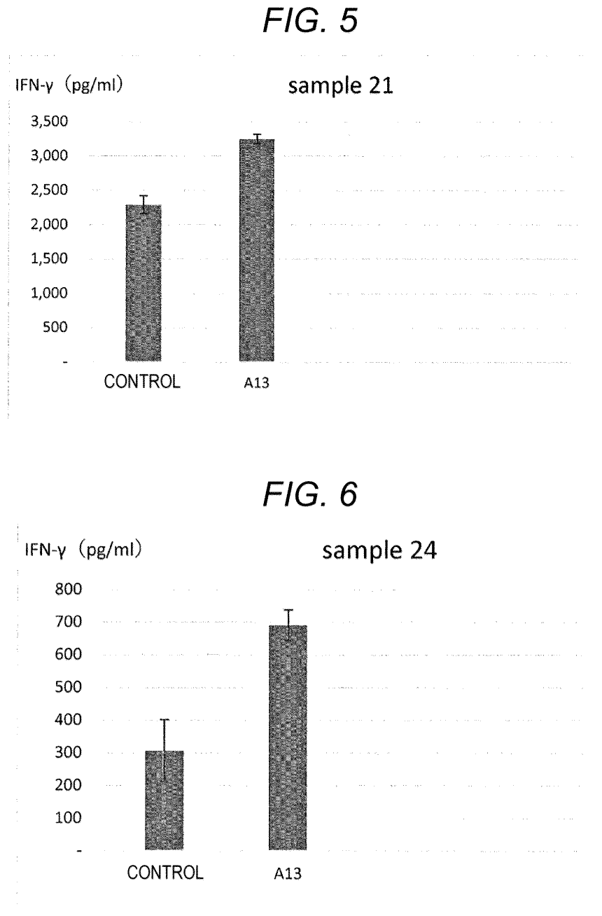

FIG. 5 is a result for a patient with HLA-A*02:01 in one of alleles (sample 21).

FIG. 6 is a result for a patient with HLA-A*24:02 in one of alleles (sample 24).

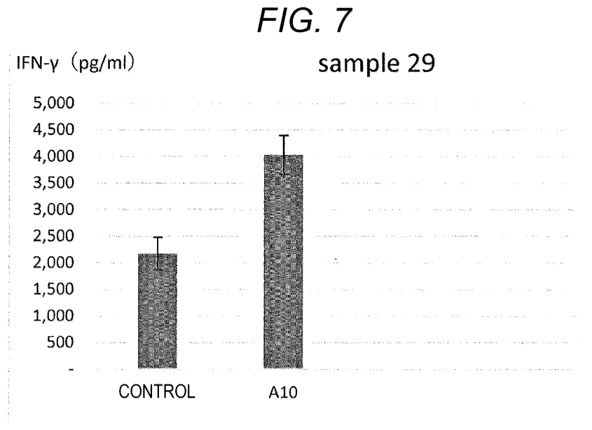

FIG. 7 is a result for a patient with HLA-A*24:02/*02:06 (sample 29).

In the drawings, “H1” means a peptide of SEQ ID NO: 47, “A15” means a peptide of SEQ ID NO: 66, “A10” means a peptide of SEQ ID NO: 16, “A19-1” means a peptide of SEQ ID NO: 20, “at-1” means a peptide of SEQ ID NO: 21, and “A13” means a peptide of SEQ ID NO: 19.

Significant production of IFN-γ was observed and immunity inducibility could be confirmed in peptides of SEQ ID NOs: 16, 19, 20, and 21 exhibiting a binding property to all of the HLA-A*24:02 molecule, the HLA-A*02:01 molecule, and the HLA-A*02:06 molecule.

In addition, significant production of IFN-γ was not observed in many of the peptides not exhibiting a binding property to all of the HLA-A*24:02 molecule, the HLA-A*02:01 molecule, and the HLA-A*02:06 molecule, but significant production of IFN-γ was observed and immunity inducibility could be confirmed in peptides of SEQ ID NOs: 47 and 66.

1-6. (canceled)

7. A method for treatment or prevention of adult T-cell leukemia in a subject, comprising a step of administrating, to the subject, a pharmaceutical composition comprising a peptide consisting of an amino acid sequence of any one of SEQ ID NOs: 1 to 68.

8. The method according to claim 7, wherein the peptide binds to one or more types of major histocompatibility antigen (HLA) molecules.

9. The method according to claim 8, wherein the peptide consists of an amino acid sequence of any one of SEQ ID NOs: 1 to 47 and binds to two or more types of HLA molecules.

10. The method according to claim 9, wherein the peptide consists of an amino acid sequence of any one of SEQ ID NOs: 1 to 33 and binds to three or more types of HLA molecules.

11. The method according to claim 7, wherein the peptide induces a cytotoxic T lymphocyte.

12. The method according to claim 11, wherein the peptide consists of an amino acid sequence of any one of SEQ ID NOs: 16, 19, 20, 21, 47, and 66.

13. The method according to claim 7, wherein the pharmaceutical composition is in a form of a vaccine.

14. A pharmaceutical composition for treatment or prevention of adult T-cell leukemia, the pharmaceutical composition comprising a peptide consisting of an amino acid sequence of any one of SEQ ID NOs: 1 to 68.

15. The pharmaceutical composition according to claim 14, wherein the peptide binds to one or more types of major histocompatibility antigen (HLA) molecules.

16. The pharmaceutical composition according to claim 15, wherein the peptide consists of an amino acid sequence of any one of SEQ ID NOs: 1 to 47 and binds to two or more types of HLA molecules.

17. The pharmaceutical composition according to claim 16, wherein the peptide consists of an amino acid sequence of any one of SEQ ID NOs: 1 to 33 and binds to three or more types of HLA molecules.

18. The pharmaceutical composition according to claim 14, wherein the peptide induces a cytotoxic T lymphocyte.

19. The pharmaceutical composition to claim 18, wherein the peptide consists of an amino acid sequence of any one of SEQ ID NOs: 16, 19, 20, 21, 47, and 66.

20. The pharmaceutical composition according to claim 14, wherein the pharmaceutical composition is in a form of a vaccine.

21. A peptide consisting of an amino acid sequence of any one of SEQ ID NOs: 1 to 68.

22. A method for producing an antigen-presenting cell having a cytotoxic T lymphocyte inducing activity, the method comprising a step of bringing the peptide according to claim 21 into contact with an antigen-presenting cell in vitro.


SEQUENCE LISTING (309 sequences)

SEQ ID NO: 1 - protein, 321 aa, Homo sapiens
MGEPQGSMRILVTGGSGLVGKAIQKVVADGAGLPGEDWVFVSSKDADLTDTAQTRALFEKVQPTHVIHLAAMVGGLFRNIKYNLDFWRKNVHMNDNVLHSAFEVGARKVVSCLSTCIFPDKTTYPIDETMIHNGPPHNSNFGYSYAKRMIDVQNRAYFQQYGCTFTAVIPTNVFGPHDNFNIEDGHVLPGLIHKVHLAKSSGSALTVWGTGNPRRQFIYSLDLAQLFIWVLREYNEVEPIILSVGEEDEVSIKEAAEAVVEAMDFHGEVTFDTTKSDGQFKKTASNSKLRTYLPDFRFTPFKQAVKETCAWFTDNYEQARK

SEQ ID NO: 2 - protein, 662 aa, Homo sapiens
AAAAARPAGGSARRWGRPGRCGLLAAGPKRVRSEPGGRLPERSLGPAHPAPAAMAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL

SEQ ID NO: 3 - protein, 797 aa, Homo sapiens
MGTLGKAREAPRKPSHGCRAASKARLEAKPANSPFPSHPSLAHITQFRMMVSLGHLAKGASLDDLIDSCIQSFDADGNLCRSNQLLQVMLTMHRIVISSAELLQKVITLYKDALAKNSPGLCLKICYFVRYWITEFWVMFKMDASLTDTMEEFQELVKAKGEELHCRLIDTTQINARDWSRKLTQRIKSNTSKKRKVSLLFDHLEPEELSEHLTYLEFKSFRRISFSDYQNYLVNSCVKENPTMERSIALCNGISQWVQLMVLSRPTPQLRAEVFIKFIQVAQKLHQLQNFNTLMAVIGGLCHSSISRLKETSSHVPHEINKVLGEMTELLSSSRNYDNYRRAYGECTDFKIPILGVHLKDLISLYEAMPDYLEDGKVNVHKLLALYNHISELVQLQEVAPPLEANKDLVHLLTLSLDLYYTEDEIYELSYAREPRNHRAPPLTPSKPPVVVDWASGVSPKPDPKTISKHVQRMVDSVFKNYDHDQDGYISQEEFEKIAASFPFSFCVMDKDREGLISRDEITAYFMRASSIYSKLGLGFPHNFQETTYLKPTFCDNCAGFLWGVIKQGYRCKDCGMNCHKQCKDLVVFECKKRAKNPVAPTENNTSVGPVSNLCSLGAKDLLHAPEEGPFTFPNGEAVEHGEESKDRTIMLMGVSSQKISLRLKRAVAHKATQTESQPWIGSEGPSGPFVLSSPRKTAQDTLYVLPSPTSPCPSPVLVRKRAFVKWENKDSLIKSKEELRHLRLPTYQELEQEINTLKADNDALKIQLKYAQKKIESLQLEKSNHVLAQMEQGDCS

SEQ ID NO: 4 - protein, 690 aa, Homo sapiens
MGSSGLGKAATLDELLCTCIEMFDDNGELDNSYLPRIVLLMHRWYLSSTELAEKLLCMYRNATGESCNEFRLKICYFMRYWILKFPAEFNLDLGLIRMTEEFREVASQLGYEKHVSLIDISSIPSYDWMRRVTQRKKVSKKGKACLLFDHLEPIELAEHLTFLEHKSFRRISFTDYQSYVIHGCLENNPTLERSIALFNGISKWVQLMVLSKPTPQQRAEVITKFINVAKKLLQLKNFNTLMAVVGGLSHSSISRLKETHSHLSSEVTKNWNEMTELVSSNGNYCNYRKAFADCDGFKIPILGVHLKDLIAVHVIFPDWTEENKVNIVKMHQLSVTLSELVSLQNASHHLEPNMDLINLLTLSLDLYHTEDDIYKLSLVLEPRNSKSQPTSPTTPNKPVVPLEWALGVMPKPDPTVINKHIRKLVESVFRNYDHDHDGYISQEDFESIAANFPFLDSFCVLDKDQDGLISKDEMMAYFLRAKSQLHCKMGPGFIHNFQEMTYLKPTFCEHCAGFLWGIIKQGYKCKDCGANCHKQCKDLLVLACRRFARAPSLSSGHGSLPGSPSLPPAQDEVFEFPGVTAGHRDLDSRAITLVTGSSRKISVRLQRATTSQATQTEPVWSEAGWGDSGSHTFPKMKSKFHDKAAKDKGFAKWENEKPRVHAGVDVVDRGTEFELDQDEGEETRQDGEDG

SEQ ID NO: 5 - protein, 673 aa, Homo sapiens
MNRKDSKRKSHQECTGKIGGRGRPRQVRRHKTCPSPREISKVMASMNLGLLSEGGCSEDELLEKCIQSFDSAGSLCHEDHMLNMVLAMHSWVLPSADLAARLLTSYQKATGDTQELRRLQICHLVRYWLMRHPEVMHQDPQLEEVIGRFWATVAREGNSAQRRLGDSSDLLSPGGPGPPLPMSSPGLGKKRKVSLLFDHLETGELAQHLTYLEFRSFQAITPQDLRSYVLQGSVRGCPALEGSVGLSNSVSRWVQVMVLSRPGPLQRAQVLDKFIHVAQRLHQLQNFNTLMAVTGGLCHSAISRLKDSHAHLSPDSTKALLELTELLASHNNYARYRRTWAGCAGFRLPVLGVHLKDLVSLHEAQPDRLPDGRLHLPKLNNLYLRLQELVALQGQHPPCSANEDLLHLLTLSLDLFYTEDEIYELSYAREPRCPKSLPPSPFNAPLVVEWAPGVTPKPDRVTLGRHVEQLVESVFKNYDPEGRGTISQEDFERLSGNFPFACHGLHPPPRQGRGSFSREELTGYLLRASAICSKLGLAFLHTFHEVTFRKPTFCDSCSGFLWGVTKQGYRCRECGLCCHKHCRDQVKVECKKRPGAKGDAGPPGAPVPSTPAPHASCGSEENHSYTLSLEPETGCQLRHAWTQTESPHPSWETDTVPCPVMDPPSTASSKLDS

SEQ ID NO: 6 - protein, 609 aa, Homo sapiens
MAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL

SEQ ID NO: 7 - protein, 671 aa, Homo sapiens
MGTQRLCGRGTQGWPGSSEQHVQEATSSAGLHSGVDELGVRSEPGGRLPERSLGPAHPAPAAMAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL

SEQ ID NO: 8 - protein, 144 aa, Homo sapiens
MAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVCVGAEHRGLGGHSVSYTICA

SEQ ID NO: 9 - protein, 610 aa, Homo sapiens
MAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPAEIREEEVQTVEDGVFDIHL

SEQ ID NO: 10 - protein, 15 aa, Homo sapiens
MGEPQGSMRILVTGG

SEQ ID NO: 11 - protein, 15 aa, Homo sapiens
VVADGAGLPGEDWVF

SEQ ID NO: 12 - protein, 15 aa, Homo sapiens
TAQTRALFEKVQPTH

SEQ ID NO: 13 - protein, 15 aa, Homo sapiens
LFRNIKYNLDFWRKN

SEQ ID NO: 14 - protein, 15 aa, Homo sapiens
VHMNDNVLHSAFEVG

SEQ ID NO: 15 - protein, 10 aa, Homo sapiens
DNVLHSAFEV

SEQ ID NO: 16 - protein, 10 aa, Homo sapiens
NVLHSAFEVG

SEQ ID NO: 17 - protein, 15 aa, Homo sapiens
NVLHSAFEVGARKVV

SEQ ID NO: 18 - protein, 10 aa, Homo sapiens
VLHSAFEVGA

SEQ ID NO: 19 - protein, 15 aa, Homo sapiens
KTTYPIDETMIHNGP

SEQ ID NO: 20 - protein, 15 aa, Homo sapiens
IHNGPPHNSNFGYSY

SEQ ID NO: 21 - protein, 15 aa, Homo sapiens
PHNSNFGYSYAKRMI

SEQ ID NO: 22 - protein, 15 aa, Homo sapiens
AYFQQYGCTFTAVIP

SEQ ID NO: 23 - protein, 15 aa, Homo sapiens
YGCTFTAVIPTNVFG

SEQ ID NO: 24 - protein, 15 aa, Homo sapiens
LFIWVLREYNEVEPI

SEQ ID NO: 25 - protein, 15 aa, Homo sapiens
LREYNEVEPIILSVG

SEQ ID NO: 26 - protein, 15 aa, Homo sapiens
EVEPIILSVGEEDEV

SEQ ID NO: 27 - protein, 15 aa, Homo sapiens
ILSVGEEDEVSIKEA

SEQ ID NO: 28 - protein, 15 aa, Homo sapiens
EEDEVSIKEAAEAVV

SEQ ID NO: 29 - protein, 15 aa, Homo sapiens
SIKEAAEAVVEAMDF

SEQ ID NO: 30 - protein, 15 aa, Homo sapiens
AEAVVEAMDFHGEVT

SEQ ID NO: 31 - protein, 15 aa, Homo sapiens
FDTTKSDGQFKKTAS

SEQ ID NO: 32 - protein, 15 aa, Homo sapiens
FRFTPFKQAVKETCA

SEQ ID NO: 33 - protein, 10 aa, Homo sapiens
LVRYWISAFP

SEQ ID NO: 34 - protein, 10 aa, Homo sapiens
FVRYWITEFW

SEQ ID NO: 35 - protein, 10 aa, Homo sapiens
FMRYWILKFP

SEQ ID NO: 36 - protein, 23 aa, Homo sapiens
LLFDHLEPMELAEHLTYLEYRSF

SEQ ID NO: 37 - protein, 28 aa, Homo sapiens
NFNTLMAVVGGLSHSSISRLKETHSHVS

SEQ ID NO: 38 - protein, 15 aa, Homo sapiens
PAAMAGTLDLDKGCT

SEQ ID NO: 39 - protein, 15 aa, Homo sapiens
DKGCTVEELLRGCIE

SEQ ID NO: 40 - protein, 15 aa, Homo sapiens
RGCIEAFDDSGKVRD

SEQ ID NO: 41 - protein, 15 aa, Homo sapiens
GKVRDPQLVRMFLMM

SEQ ID NO: 42 - protein, 15 aa, Homo sapiens
MFLMMHPWYIPSSQL

SEQ ID NO: 43 - protein, 15 aa, Homo sapiens
PSSQLAAKLLHIYQQ

SEQ ID NO: 44 - protein, 15 aa, Homo sapiens
HIYQQSRKDNSNSLQ

SEQ ID NO: 45 - protein, 15 aa, Homo sapiens
SNSLQVKTCHLVRYW

SEQ ID NO: 46 - protein, 15 aa, Homo sapiens
LVRYWISAFPAEFDL

SEQ ID NO: 47 - protein, 15 aa, Homo sapiens
AEFDLNPELAEQIKE

SEQ ID NO: 48 - protein, 15 aa, Homo sapiens
EQIKELKALLDQEGN

SEQ ID NO: 49 - protein, 15 aa, Homo sapiens
DQEGNRRHSSLIDID

SEQ ID NO: 50 - protein, 15 aa, Homo sapiens
LIDIDSVPTYKWKRQ

SEQ ID NO: 51 - protein, 15 aa, Homo sapiens
KWKRQVTQRNPVGQK

SEQ ID NO: 52 - protein, 15 aa, Homo sapiens
PVGQKKRKMSLLFDH

SEQ ID NO: 53 - protein, 15 aa, Homo sapiens
LLFDHLEPMELAEHL

SEQ ID NO: 54 - protein, 15 aa, Homo sapiens
LAEHLTYLEYRSFCK

SEQ ID NO: 55 - protein, 15 aa, Homo sapiens
RSFCKILFQDYHSFV

SEQ ID NO: 56 - protein, 15 aa, Homo sapiens
YHSFVTHGCTVDNPV

SEQ ID NO: 57 - protein, 15 aa, Homo sapiens
VDNPVLERFISLFNS

SEQ ID NO: 58 - protein, 15 aa, Homo sapiens
SLFNSVSQWVQLMIL

SEQ ID NO: 59 - protein, 15 aa, Homo sapiens
QLMILSKPTAPQRAL

SEQ ID NO: 60 - protein, 15 aa, Homo sapiens
PQRALVITHFVHVAE

SEQ ID NO: 61 - protein, 15 aa, Homo sapiens
VHVAEKLLQLQNFNT

SEQ ID NO: 62 - protein, 15 aa, Homo sapiens
QNFNTLMAVVGGLSH

SEQ ID NO: 63 - protein, 15 aa, Homo sapiens
GGLSHSSISRLKETH

SEQ ID NO: 64 - protein, 15 aa, Homo sapiens
LKETHSHVSPETIKL

SEQ ID NO: 65 - protein, 15 aa, Homo sapiens
ETIKLWEGLTELVTA

SEQ ID NO: 66 - protein, 15 aa, Homo sapiens
ELVTATGNYGNYRRR

SEQ ID NO: 67 - protein, 15 aa, Homo sapiens
NYRRRLAACVGFRFP

SEQ ID NO: 68 - protein, 15 aa, Homo sapiens
GFRFPILGVHLKDLV

SEQ ID NO: 69 - protein, 15 aa, Homo sapiens
LKDLVALQLALPDWL

SEQ ID NO: 70 - protein, 15 aa, Homo sapiens
LPDWLDPARTRLNGA

SEQ ID NO: 71 - protein, 15 aa, Homo sapiens
RLNGAKMKQLFSILE

SEQ ID NO: 72 - protein, 15 aa, Homo sapiens
FSILEELAMVTSLRP

SEQ ID NO: 73 - protein, 15 aa, Homo sapiens
TSLRPPVQANPDLLS

SEQ ID NO: 74 - protein, 15 aa, Homo sapiens
PDLLSLLTVSLDQYQ

SEQ ID NO: 75 - protein, 15 aa, Homo sapiens
LDQYQTEDELYQLSL

SEQ ID NO: 76 - protein, 15 aa, Homo sapiens
YQLSLQREPRSKSSP

SEQ ID NO: 77 - protein, 15 aa, Homo sapiens
SKSSPTSPTSCTPPP

SEQ ID NO: 78 - protein, 15 aa, Homo sapiens
CTPPPRPPVLEEWTS

SEQ ID NO: 79 - protein, 15 aa, Homo sapiens
EEWTSAAKPKLDQAL

SEQ ID NO: 80 - protein, 15 aa, Homo sapiens
LDQALVVEHIEKMVE

SEQ ID NO: 81 - protein, 15 aa, Homo sapiens
EKMVESVFRNFDVDG

SEQ ID NO: 82 - protein, 15 aa, Homo sapiens
FDVDGDGHISQEEFQ

SEQ ID NO: 83 - protein, 15 aa, Homo sapiens
QEEFQIIRGNFPYLS

SEQ ID NO: 84 - protein, 15 aa, Homo sapiens
FPYLSAFGDLDQNQD

SEQ ID NO: 85 - protein, 15 aa, Homo sapiens
DQNQDGCISREEMVS

SEQ ID NO: 86 - protein, 15 aa, Homo sapiens
EEMVSYFLRSSSVLG

SEQ ID NO: 87 - protein, 15 aa, Homo sapiens
SSVLGGRMGFVHNFQ

SEQ ID NO: 88 - protein, 15 aa, Homo sapiens
VHNFQESNSLRPVAC

SEQ ID NO: 89 - protein, 15 aa, Homo sapiens
RPVACRHCKALILGI

SEQ ID NO: 90 - protein, 15 aa, Homo sapiens
LILGIYKQGLKCRAC

SEQ ID NO: 91 - protein, 15 aa, Homo sapiens
KCRACGVNCHKQCKD

SEQ ID NO: 92 - protein, 15 aa, Homo sapiens
KQCKDRLSVECRRRA

SEQ ID NO: 93 - protein, 15 aa, Homo sapiens
CRRRAQSVSLEGSAP

SEQ ID NO: 94 - protein, 15 aa, Homo sapiens
EGSAPSPSPMHSHHH

SEQ ID NO: 95 - protein, 15 aa, Homo sapiens
HSHHHRAFSFSLPRP

SEQ ID NO: 96 - protein, 15 aa, Homo sapiens
SLPRPGRRGSRPPEI

SEQ ID NO: 97 - protein, 15 aa, Homo sapiens
RPPEIREEEVQTVED

SEQ ID NO: 98 - protein, 15 aa, Homo sapiens
EEVQTVEDGVFDIHL

SEQ ID NO: 99 - protein, 7 aa, Homo sapiens
  SITE 4: note = misc_feature - Xaa can be any naturally occurring amino acid
LHSXFEV

SEQ ID NO: 100 - protein, 8 aa, Homo sapiens
VLHSAFEV

SEQ ID NO: 101 - protein, 10 aa, Homo sapiens
RKLHSFYEVK

SEQ ID NO: 102 - protein, 10 aa, Homo sapiens
LKLHSRFYEL

SEQ ID NO: 103 - protein, 10 aa, Homo sapiens
QKKHSGFEDE

SEQ ID NO: 104 - protein, 10 aa, Homo sapiens
LALHSVFEGL

SEQ ID NO: 105 - protein, 10 aa, Homo sapiens
RRLHSPPEVE

SEQ ID NO: 106 - protein, 10 aa, Homo sapiens
LSLHRMFEVV

SEQ ID NO: 107 - protein, 10 aa, Homo sapiens
QKLHLHFERL

SEQ ID NO: 108 - protein, 10 aa, Homo sapiens
QRLHLHFEKV

SEQ ID NO: 109 - protein, 10 aa, Homo sapiens
LLLHSIFELN

SEQ ID NO: 110 - protein, 10 aa, Homo sapiens
NGLHSHSEVL

SEQ ID NO: 111 - protein, 10 aa, Homo sapiens
RRLHLHFERV

SEQ ID NO: 112 - protein, 10 aa, Homo sapiens
LRLHSPPEVT

SEQ ID NO: 113 - protein, 10 aa, Homo sapiens
SHLHSLFRVL

SEQ ID NO: 114 - protein, 10 aa, Homo sapiens
VELHSHSEVP

SEQ ID NO: 115 - protein, 10 aa, Homo sapiens
GHLHSQLEVS

SEQ ID NO: 116 - protein, 10 aa, Homo sapiens
EKKVSSFEVF

SEQ ID NO: 117 - protein, 10 aa, Homo sapiens
DGLHSSNEVL

SEQ ID NO: 118 - protein, 10 aa, Homo sapiens
LSLHSLFELR

SEQ ID NO: 119 - protein, 10 aa, Homo sapiens
FTPHSRFEVL

SEQ ID NO: 120 - protein, 10 aa, Homo sapiens
IKLPSSFEKW

SEQ ID NO: 121 - protein, 10 aa, Homo sapiens
VMLLSLFEEE

SEQ ID NO: 122 - protein, 10 aa, Homo sapiens
FALHSLFEAK

SEQ ID NO: 123 - protein, 10 aa, Homo sapiens
RKLKASFEVS

SEQ ID NO: 124 - protein, 10 aa, Homo sapiens
KALHSDFIVK

SEQ ID NO: 125 - protein, 10 aa, Homo sapiens
KKLRSSFESS

SEQ ID NO: 126 - protein, 10 aa, Homo sapiens
LQLHSPFERG

SEQ ID NO: 127 - protein, 10 aa, Homo sapiens
RKCHSSGEVQ

SEQ ID NO: 128 - protein, 10 aa, Homo sapiens
RKLLSDFPVV

SEQ ID NO: 129 - protein, 10 aa, Homo sapiens
KKRHSYFEKP

SEQ ID NO: 130 - protein, 10 aa, Homo sapiens
VHLHSFFAVG

SEQ ID NO: 131 - protein, 10 aa, Homo sapiens
EALLSGFEVI

SEQ ID NO: 132 - protein, 10 aa, Homo sapiens
EKLFSQFEVD

SEQ ID NO: 133 - protein, 10 aa, Homo sapiens
GDLKSGFEEV

SEQ ID NO: 134 - protein, 10 aa, Homo sapiens
KGLHSKKEVP

SEQ ID NO: 135 - protein, 10 aa, Homo sapiens
EFLASSFEVS

SEQ ID NO: 136 - protein, 10 aa, Homo sapiens
GVLHSKFWVV

SEQ ID NO: 137 - protein, 10 aa, Homo sapiens
KQLVHHFEVD

SEQ ID NO: 138 - protein, 10 aa, Homo sapiens
RHLHSVLEEL

SEQ ID NO: 139 - protein, 10 aa, Homo sapiens
RLLSSGFEEV

SEQ ID NO: 140 - protein, 10 aa, Homo sapiens
QMLHPIFEEA

SEQ ID NO: 141 - protein, 10 aa, Homo sapiens
RRLHSTHEEL

SEQ ID NO: 142 - protein, 10 aa, Homo sapiens
RKLHWLFELL

SEQ ID NO: 143 - protein, 10 aa, Homo sapiens
VELHSDMEVT

SEQ ID NO: 144 - protein, 10 aa, Homo sapiens
RKLLHRFETE

SEQ ID NO: 145 - protein, 10 aa, Homo sapiens
NKLISEFEEE

SEQ ID NO: 146 - protein, 10 aa, Homo sapiens
SKLVSNKEVV

SEQ ID NO: 147 - protein, 10 aa, Homo sapiens
LLLHSLFPVP

SEQ ID NO: 148 - protein, 10 aa, Homo sapiens
RKLDSVFEER

SEQ ID NO: 149 - protein, 10 aa, Homo sapiens
RFKHSSTEVL

SEQ ID NO: 150 - protein, 10 aa, Homo sapiens
RRKLHSFYEV

SEQ ID NO: 151 - protein, 10 aa, Homo sapiens
RKLLSSGFEI

SEQ ID NO: 152 - protein, 10 aa, Homo sapiens
RRLLSSGFEE

SEQ ID NO: 153 - protein, 10 aa, Homo sapiens
RKKLHKFEET

SEQ ID NO: 154 - protein, 10 aa, Homo sapiens
LKKLHDFEEQ

SEQ ID NO: 155 - protein, 10 aa, Homo sapiens
GLLLHSIFEL

SEQ ID NO: 156 - protein, 10 aa, Homo sapiens
QKKLHDFEEQ

SEQ ID NO: 157 - protein, 10 aa, Homo sapiens
QKKLSSGEEK

SEQ ID NO: 158 - protein, 10 aa, Homo sapiens
RKHLHSGQEA

SEQ ID NO: 159 - protein, 10 aa, Homo sapiens
QKLVHSLFES

SEQ ID NO: 160 - protein, 10 aa, Homo sapiens
CYLLHSFEEF

SEQ ID NO: 161 - protein, 10 aa, Homo sapiens
SKTLHSVEEK

SEQ ID NO: 162 - protein, 10 aa, Homo sapiens
LKRLHEFEEQ

SEQ ID NO: 163 - protein, 10 aa, Homo sapiens
VKELHSAEEG

SEQ ID NO: 164 - protein, 10 aa, Homo sapiens
SLLLHSQEEK

SEQ ID NO: 165 - protein, 10 aa, Homo sapiens
AVHLHSGEEL

SEQ ID NO: 166 - protein, 10 aa, Homo sapiens
HTRLHSGEEP

SEQ ID NO: 167 - protein, 10 aa, Homo sapiens
MPLLHAIFEV

SEQ ID NO: 168 - protein, 10 aa, Homo sapiens
EKKRHSYFEK

SEQ ID NO: 169 - protein, 10 aa, Homo sapiens
QAKLHSQEED

SEQ ID NO: 170 - protein, 10 aa, Homo sapiens
QQKKHSGFED

SEQ ID NO: 171 - protein, 10 aa, Homo sapiens
LKKLHQQFEM

SEQ ID NO: 172 - protein, 10 aa, Homo sapiens
KLLLHSGVEN

SEQ ID NO: 173 - protein, 10 aa, Homo sapiens
FSKLHTFEEV

SEQ ID NO: 174 - protein, 10 aa, Homo sapiens
QAKLSSFEET

SEQ ID NO: 175 - protein, 10 aa, Homo sapiens
SLALHSVFEG

SEQ ID NO: 176 - protein, 10 aa, Homo sapiens
EFALHSLFEA

SEQ ID NO: 177 - protein, 10 aa, Homo sapiens
MRHLKSFFEA

SEQ ID NO: 178 - protein, 10 aa, Homo sapiens
QKKRHSFLES

SEQ ID NO: 179 - protein, 10 aa, Homo sapiens
QKKLHHFFIG

SEQ ID NO: 180 - protein, 10 aa, Homo sapiens
TLSLHSLFEL

SEQ ID NO: 181 - protein, 10 aa, Homo sapiens
LRLFHSFEEL

SEQ ID NO: 182 - protein, 10 aa, Homo sapiens
QKFLHSSFVA

SEQ ID NO: 183 - protein, 10 aa, Homo sapiens
AWLLHSGPEG

SEQ ID NO: 184 - protein, 10 aa, Homo sapiens
TKLLHRTEEL

SEQ ID NO: 185 - protein, 10 aa, Homo sapiens
FKTLSSKFEL

SEQ ID NO: 186 - protein, 10 aa, Homo sapiens
KRRLHLVFEY

SEQ ID NO: 187 - protein, 10 aa, Homo sapiens
EHLLNSGFEV

SEQ ID NO: 188 - protein, 10 aa, Homo sapiens
VMKLLSIFES

SEQ ID NO: 189 - protein, 10 aa, Homo sapiens
KLELHSSEEA

SEQ ID NO: 190 - protein, 10 aa, Homo sapiens
KLPLHSSEEA

SEQ ID NO: 191 - protein, 10 aa, Homo sapiens
LKRLLSNEEE

SEQ ID NO: 192 - protein, 10 aa, Homo sapiens
TTRLHSLEEK

SEQ ID NO: 193 - protein, 10 aa, Homo sapiens
HARLHSLEET

SEQ ID NO: 194 - protein, 10 aa, Homo sapiens
LPRLHGHFEQ

SEQ ID NO: 195 - protein, 10 aa, Homo sapiens
VKTLHHKEEV

SEQ ID NO: 196 - protein, 10 aa, Homo sapiens
SPSLHSREEA

SEQ ID NO: 197 - protein, 10 aa, Homo sapiens
PRKLHWLFEL

SEQ ID NO: 198 - protein, 10 aa, Homo sapiens
LKKLLSFAEN

SEQ ID NO: 199 - protein, 12 aa, Homo sapiens
ILVTGGSGLVGK

SEQ ID NO: 200 - protein, 19 aa, Homo sapiens
VVADGAGLPGEDWVFVSSK

SEQ ID NO: 201 - protein, 11 aa, Homo sapiens
DADLTDTAQTR

SEQ ID NO: 202 - protein, 18 aa, Homo sapiens
VQPTHVIHLAAMVGGLFR

SEQ ID NO: 203 - protein, 7 aa, Homo sapiens
YNLDFWR

SEQ ID NO: 204 - protein, 8 aa, Homo sapiens
YNLDFWRK

SEQ ID NO: 205 - protein, 18 aa, Homo sapiens
NVHMNDNVLHSAFEVGAR

SEQ ID NO: 206 - protein, 19 aa, Homo sapiens
NVHMNDNVLHSAFEVGARK

SEQ ID NO: 207 - protein, 13 aa, Homo sapiens
VVSCLSTCIFPDK

SEQ ID NO: 208 - protein, 7 aa, Homo sapiens
MIDVQNR

SEQ ID NO: 209 - protein, 8 aa, Homo sapiens
RMIDVQNR

SEQ ID NO: 210 - protein, 15 aa, Homo sapiens
SSGSALTVWGTGNPR

SEQ ID NO: 211 - protein, 16 aa, Homo sapiens
SSGSALTVWGTGNPRR

SEQ ID NO: 212 - protein, 26 aa, Homo sapiens
TTYPIDETMIHNGPPHNSNFGYSYAK

SEQ ID NO: 213 - protein, 21 aa, Homo sapiens
EYNEVEPIILSVGEEDEVSIK

SEQ ID NO: 214 - protein, 7 aa, Homo sapiens
TYLPDFR

SEQ ID NO: 215 - protein, 9 aa, Homo sapiens
LRTYLPDFR

SEQ ID NO: 216 - protein, 15 aa, Homo sapiens
GSMRILVTGGSGLVG

SEQ ID NO: 217 - protein, 15 aa, Homo sapiens
LVTGGSGLVGKAIQK

SEQ ID NO: 218 - protein, 15 aa, Homo sapiens
SGLVGKAIQKVVADG

SEQ ID NO: 219 - protein, 15 aa, Homo sapiens
KAIQKVVADGAGLPG

SEQ ID NO: 220 - protein, 15 aa, Homo sapiens
AGLPGEDWVFVSSKD

SEQ ID NO: 221 - protein, 15 aa, Homo sapiens
EDWVFVSSKDADLTD

SEQ ID NO: 222 - protein, 15 aa, Homo sapiens
VSSKDADLTDTAQTR

SEQ ID NO: 223 - protein, 15 aa, Homo sapiens
ADLTDTAQTRALFEK

SEQ ID NO: 224 - protein, 15 aa, Homo sapiens
ALFEKVQPTHVIHLA

SEQ ID NO: 225 - protein, 15 aa, Homo sapiens
VQPTHVIHLAAMVGG

SEQ ID NO: 226 - protein, 15 aa, Homo sapiens
VIHLAAMVGGLFRNI

SEQ ID NO: 227 - protein, 15 aa, Homo sapiens
AMVGGLFRNIKYNLD

SEQ ID NO: 228 - protein, 15 aa, Homo sapiens
KYNLDFWRKNVHMND

SEQ ID NO: 229 - protein, 15 aa, Homo sapiens
FWRKNVHMNDNVLHS

SEQ ID NO: 230 - protein, 15 aa, Homo sapiens
NVLHSAFEVGARKVV

SEQ ID NO: 231 - protein, 15 aa, Homo sapiens
AFEVGARKVVSCLST

SEQ ID NO: 232 - protein, 15 aa, Homo sapiens
ARKVVSCLSTCIFPD

SEQ ID NO: 233 - protein, 15 aa, Homo sapiens
SCLSTCIFPDKTTYP

SEQ ID NO: 234 - protein, 15 aa, Homo sapiens
CIFPDKTTYPIDETM

SEQ ID NO: 235 - protein, 15 aa, Homo sapiens
IDETMIHNGPPHNSN

SEQ ID NO: 236 - protein, 15 aa, Homo sapiens
PHNSNFGYSYAKRMI

SEQ ID NO: 237 - protein, 15 aa, Homo sapiens
FGYSYAKRMIDVQNR

SEQ ID NO: 238 - protein, 15 aa, Homo sapiens
AKRMIDVQNRAYFQQ

SEQ ID NO: 239 - protein, 15 aa, Homo sapiens
DVQNRAYFQQYGCTF

SEQ ID NO: 240 - protein, 15 aa, Homo sapiens
TAVIPTNVFGPHDNF

SEQ ID NO: 241 - protein, 15 aa, Homo sapiens
TNVFGPHDNFNIEDG

SEQ ID NO: 242 - protein, 15 aa, Homo sapiens
PHDNFNIEDGHVLPG

SEQ ID NO: 243 - protein, 15 aa, Homo sapiens
NIEDGHVLPGLIHKV

SEQ ID NO: 244 - protein, 15 aa, Homo sapiens
HVLPGLIHKVHLAKS

SEQ ID NO: 245 - protein, 15 aa, Homo sapiens
LIHKVHLAKSSGSAL

SEQ ID NO: 246 - protein, 15 aa, Homo sapiens
HLAKSSGSALTVWGT

SEQ ID NO: 247 - protein, 15 aa, Homo sapiens
SGSALTVWGTGNPRR

SEQ ID NO: 248 - protein, 15 aa, Homo sapiens
TVWGTGNPRRQFIYS

SEQ ID NO: 249 - protein, 15 aa, Homo sapiens
GNPRRQFIYSLDLAQ

SEQ ID NO: 250 - protein, 15 aa, Homo sapiens
QFIYSLDLAQLFIWV

SEQ ID NO: 251 - protein, 15 aa, Homo sapiens
LDLAQLFIWVLREYN

SEQ ID NO: 252 - protein, 15 aa, Homo sapiens
LFIWVLREYNEVEPI

SEQ ID NO: 253 - protein, 15 aa, Homo sapiens
EAMDFHGEVTFDTTK

SEQ ID NO: 254 - protein, 15 aa, Homo sapiens
HGEVTFDTTKSDGQF

SEQ ID NO: 255 - protein, 15 aa, Homo sapiens
SDGQFKKTASNSKLR

SEQ ID NO: 256 - protein, 15 aa, Homo sapiens
KKTASNSKLRTYLPD

SEQ ID NO: 257 - protein, 15 aa, Homo sapiens
NSKLRTYLPDFRFTP

SEQ ID NO: 258 - protein, 15 aa, Homo sapiens
TYLPDFRFTPFKQAV

SEQ ID NO: 259 - protein, 15 aa, Homo sapiens
FKQAVKETCAWFTDN

SEQ ID NO: 260 - protein, 16 aa, Homo sapiens
KETCAWFTDNYEQARK

SEQ ID NO: 261 - protein, 20 aa, Homo sapiens
GQFRVIGPRHPIRALVGDEV

SEQ ID NO: 262 - protein, 21 aa, Homo sapiens
MEVGWYRPPFSRVVHLYRNGK

SEQ ID NO: 263 - protein, 20 aa, Homo sapiens
KYLATASTMDHARHGFLPRH

SEQ ID NO: 264 - protein, 17 aa, Homo sapiens
ENPVVHFFKNIVTPRTP

SEQ ID NO: 265 - protein, 19 aa, Homo sapiens
LSRFSWGAEGQRPGFGYGG

SEQ ID NO: 266 - protein, 25 aa, Homo sapiens
AQGTLSKIFKLGGRDSRSGSPMARR

SEQ ID NO: 267 - protein, 16 aa, Homo sapiens
HCLGKWLGHPDKFVGI

SEQ ID NO: 268 - protein, 11 aa, Influenza A virus
FVFTLTVPSER

SEQ ID NO: 269 - protein, 15 aa, Influenza A virus
SGPLKAEIAQRLEDV

SEQ ID NO: 270 - protein, 16 aa, Influenza A virus
YDVPDYASLRSLVASS

SEQ ID NO: 271 - protein, 13 aa, Influenza B virus
PYYTGEHAKAIGN

SEQ ID NO: 272 - protein, 12 aa, Clostridium tetani
GQIGNDPNRDIL

SEQ ID NO: 273 - protein, 12 aa, Influenza A virus
PKYVKQNTLKLA

SEQ ID NO: 274 - protein, 13 aa, Influenza A virus
PKYVKQNTLKLAT

SEQ ID NO: 275 - protein, 9 aa, Influenza A virus
DRLRRDQKS

SEQ ID NO: 276 - protein, 19 aa, Human herpesvirus 4
AGLTLSLLVICSYLFISRG

SEQ ID NO: 277 - protein, 15 aa, Clostridium tetani
QYIKANSKFIGITEL

SEQ ID NO: 278 - protein, 14 aa, Clostridium tetani
QYIKANSKFIGITE

SEQ ID NO: 279 - protein, 21 aa, Clostridium tetani
FNNFTVSFWLRVPKVSASHLE

SEQ ID NO: 280 - protein, 13 aa, Human herpesvirus 4
TSLYNLRRGTALA

SEQ ID NO: 281 - protein, 16 aa, Clostridium tetani
KFIIKRYTPNNEIDSF

SEQ ID NO: 282 - protein, 16 aa, Clostridium tetani
VSIDKFRIFCKALNPK

SEQ ID NO: 283 - protein, 18 aa, Human herpesvirus 4
VPGLYSPCRAFFNKEELL

SEQ ID NO: 284 - protein, 14 aa, Human cytomegalovirus
DKREMWMACIKELH

SEQ ID NO: 285 - protein, 15 aa, Human herpesvirus 4
TGHGARTSTEPTTDY

SEQ ID NO: 286 - protein, 13 aa, Human herpesvirus 4
KELKRQYEKKLRQ

SEQ ID NO: 287 - protein, 16 aa, Influenza A virus
RGYFKMRTGKSSIMRS

SEQ ID NO: 288 - protein, 11 aa, Human herpesvirus 4
TVFYNIPPMPL

SEQ ID NO: 289 - protein, 15 aa, Human herpesvirus 4
AEGLRALLARSHVER

SEQ ID NO: 290 - protein, 20 aa, Human herpesvirus 4
PGPLRESIVCYFMVFLQTHI

SEQ ID NO: 291 - protein, 26 aa, Homo sapiens
DLLHAPEEGPFTFPNGEAVEHGEESK

SEQ ID NO: 292 - protein, 11 aa, Homo sapiens
IVISSAELLQK

SEQ ID NO: 293 - protein, 23 aa, Homo sapiens
VSLLFDHLEPEELSEHLTYLEFK

SEQ ID NO: 294 - protein, 11 aa, Homo sapiens
AFSFSLPRPGR

SEQ ID NO: 295 - protein, 8 aa, Homo sapiens
ALILGIYK

SEQ ID NO: 296 - protein, 10 aa, Homo sapiens
ALLDQEGNRR

SEQ ID NO: 297 - protein, 13 aa, Homo sapiens
ALVITHFVHVAEK

SEQ ID NO: 298 - protein, 17 aa, Homo sapiens
DLVALQLALPDWLDPAR

SEQ ID NO: 299 - protein, 9 aa, Homo sapiens
DNSNSLQVK

SEQ ID NO: 300 - protein, 14 aa, Homo sapiens
HSSLIDIDSVPTYK

SEQ ID NO: 301 - protein, 10 aa, Homo sapiens
KDNSNSLQVK

SEQ ID NO: 302 - protein, 12 aa, Homo sapiens
LDQALVVEHIEK

SEQ ID NO: 303 - protein, 9 aa, Homo sapiens
LLHIYQQSR

SEQ ID NO: 304 - protein, 24 aa, Homo sapiens
LLQLQNFNTLMAVVGGLSHSSISR

SEQ ID NO: 305 - protein, 19 aa, Homo sapiens
LWEGLTELVTATGNYGNYR

SEQ ID NO: 306 - protein, 18 aa, Homo sapiens
MFLMMHPWYIPSSQLAAK

SEQ ID NO: 307 - protein, 8 aa, Homo sapiens
VRDPQLVR

SEQ ID NO: 308 - protein, 21 aa, Homo sapiens
YWISAFPAEFDLNPELAEQIK

SEQ ID NO: 309 - protein, 8 aa, Homo sapiens
LSLVLEPR